(19) United States
(12) Patent Application Publication
(10) Pub. No.: US 2018/0050068 A1
(43) Pub. Date: Feb. 22, 2018

(54) METASTATIC CANCER DECOY TRAP

(71) Applicant: Case Western Reserve University, Cleveland, OH (US)

(72) Inventors: Diego Correa, Cleveland, OH (US); Arnold I. Caplan, Cleveland, OH (US)

(21) Appl. No.: 15/716,893

(22) Filed: Sep. 27, 2017

Related U.S. Application Data
(63) Continuation-in-part of application No. 15/327,332, filed on Jan. 18, 2017.
(60) Provisional application No. 62/026,081, filed on Jul. 18, 2014.

(51) Int. Cl. A61K 35/44; A61K 47/46; A61K 47/02; G01N 33/574; A61K 49/00

(52) U.S. Cl. CPC A61K 35/44; A61K 47/46; A61K 47/02; G01N 33/57423; A61K 49/0002; G01N 33/57415; G01N 33/57434; G01N 33/574

(57) ABSTRACT

A method of treating or preventing cancer metastasis in a subject is described. The method includes implanting a decoy trap within the subject, the decoy trap comprising an implantable trap device including a metastatic cancer attractant. Methods of diagnosing cancer metastasis in a subject using the decoy traps are also described. The methods include implanting the decoy trap in the subject and allowing the decoy trap to remain within the subject for a period of time, and then diagnosing the subject as having metastatic cancer if metastatic cancer cells are detected in the decoy trap.

This application is a continuation-in-part of U.S. patent application Ser. No. 15/327,332, filed on Jan. 18, 2017, which was a national stage application claiming the benefit of International Patent Application No. PCT/US2015/041061, filed on Jul. 20, 2015, which claimed the benefit of the benefit of U.S. Provisional Application Ser. No. 62/026,081 filed Jul. 18, 2014, all of which are incorporated by reference herein.

### GOVERNMENT FUNDING

This invention was made with government support under grant No. RO1 CA163562-01A1 awarded by the National Cancer Institute of the National Institutes of Health. The government has certain rights in this invention.

### BACKGROUND

Mesenchymal Stem Cells (MSC) have long been described as cellular progenitors of mesenchymal lineages including bone, cartilage, fat, muscle and other connective tissues. In addition, a secretory capacity has been identified with both immunomodulatory and trophic activities, exerted at sites of injury where they interact with other local cellular components. The recent identification of the MSC niche in the perivascular space as pericytes has opened the possibility that MSC may have additional roles directly controlling tissue homeostasis from their cardinal abluminal location. Caplan, A. I., Cell Stem Cell 2008; 3(3), 229-230.

Metastasis is a leading prognostic indicator for cancer survival and a major contributor to cancer mortality. The skeleton and liver are common organs for cancer dissemination in various malignancies including malignant melanoma. Although the preponderance of distant metastases implies a selective advantage for arriving disseminating cells, recent studies have determined that the rates of tumor growth, invasion, and metastasis are in fact independent pathological traits governed by different sets of genes. Nguyen D X, Massague J, Nat Rev Genet 2007; 8(5), 341-352. Nevertheless, they share a common feature, namely their dependence on the vasculature that provides access to oxygen and nutrients, as well as a route for cancer cell dissemination. Current cancer therapies are designed to alter not only specific biological functions in cancer cells, but also to target components of the tumor microenvironment (TME)/stroma, especially the vasculature.

Pericytes are a specialized cell type that function abluminally covering and stabilizing blood vessels following their recruitment to forming vessels as progenitor cells via PDGF-B/PDGFRB signaling. Armulik et al., Circ Res 2005; 97(6), 512-523. Pericyte involvement in primary tumors growth constitutes a novel therapeutic target based on compelling evidence showing superior reduction in size when targeted in parallel with endothelial cells (ECs). Bergers et al., J Clin Invest 2003; 111(9),1287-1295. However, pericyte coverage of the vasculature seems to differentially regulate tumor growth and metastatic potential, as intravasation of cancer cells is increased in primary tumors grown in mice with a genetically-determined deficient pericyte coverage. Xian et al., J Clin Invest 2006; 116(3), 642-651. These findings have led to the appreciation, in some cases, of pericytes in primary tumors as gatekeepers for cancer dissemination. Gerhardt H, Semb H., J Mol Med 2008; 86(2),135-144. In parallel, in response to tumor signals, BM-derived progenitor cells of mesenchymal origin (as MSCs) are recruited to the tumor stroma localizing in perivascular sites and helping to assemble a supporting vascular network critical for tumor growth. Bergfeld S A, DeClerck Y A., Cancer metastasis reviews 2010; 29(2), 249-261. Taken together, BM-derived MSC (BM-MSC) appear to play a critical role during primary tumor formation, and growth.

In contrast, the role of pericytes at the target organ microenvironment during cancer cell extravasation is not known. Recently, data suggest that cellular and molecular elements in the BM are related to the establishment and progression of skeletal metastasis. For example, blocking PDGFB with a multi-target tyrosine kinase inhibitor (Sunitinib) impairs bone invasion of circulating osteotropic lung cancer cell lines due to altered tumor cell-BM stroma interactions. Catena et al., Cancer Research 2011; 71(1), 164-174. In addition, it has been established that invading cancer cells physically associate with mesenchymal derived cells in the BM affecting various biological activities of engrafted cancer cells, including dormancy/quiescence, resistance to chemotherapy and metastatic growth. Corcoran et al., PLoS One 2008; 3(6), e2563. However, the specific identity of the cellular and molecular elements, as well as the precise location where the sequence of events occur during extravasation is still not well understood.

The mechanisms governing skeletal metastasis involve comparable details to those used by hematopoietic stem cells (HSC) and their progeny entering the BM. The “homing” behavior of HSCs and exit of progeny relies on the existence of a specific physical niche within the BM where other cellular players, including MSC, favor the constant trafficking of such progenitors across the sinusoidal wall. Shiozawa et al showed that invading osteotropic prostate cancer cells enter the HSC niche competing with resident cells and thus establishing physical anchors for further growth inside the BM. Shiozawa et al., The Journal of clinical investigation, 2011; 121(4), 1298-1312.

There remains a need for novel methods of diagnosing and treating metastatic cancer, such as bone metastasis.

### SUMMARY

With the notion of BM-MSC as pericytes, the inventors propose taking advantage of the physical interaction of invading cancer cells and resident BM-MSC occurring at the abluminal space of BM sinusoids as a determinant step in the initiation and fate of skeletal metastasis. The inventors have shown that altering the physical interaction between vascular components of the target organ microenvironment (ECs and MSC/pericytes) via genetic manipulation of PDGF-B, dramatically impairs the engraftment of intraarterially-delivered cancer cells, thus reducing the frequency of osteolytic bone metastasis. Through in vitro and in vivo approaches, including a humanized assay in which fully functional extraskeletal bone is engineered with human MSC (hMSC), the essential molecular players and mechanisms involved in the extravasation of circulating MCC to the BM were established, that become disrupted in the absence of sinusoidal MSC/pericytes. In parallel, they observed that the situation in the BM is replicated in the liver exclusively, whereas no invasion by melanoma was seen in mutant mice. The inventors therefore propose that the presence of MSC as pericytes surrounding BM and liver sinusoids is required for extravasation of MCC, and that the effects of the EC/pericyte dissociation at the metastatic target organ do not mirror its effects during intravasation at the primary tumor.

The inventors show that MSC/pericytes function as sentinels regulating cancer cell dissemination with a differential effect during intravasation at the primary tumor and extravasation at the target organ. The molecular mechanisms, cellular players and locations during the establishment of melanoma metastasis to BM and liver were defined. This can be used to trap metastatic cancer cells in order to treat and/or diagnose metastatic cancer.

Accordingly, in one aspect, the invention provides a method of treating cancer metastasis in a subject, comprising implanting a decoy trap within the subject, the decoy trap comprising an implantable trap device including a metastatic cancer attractant. In some embodiments, the method is used to treat the metastasis originating from breast cancer, prostate cancer, or lung cancer primary tumors.

In one embodiment, the cancer metastasis is bone metastasis, and the metastatic cancer attractant is a bone-seeking cancer attractant. In another embodiment, the bone-seeking cancer attractant is pericytes. In a further embodiment, the subject is human and the pericytes are human pericytes.

In some embodiments, the implantable trap device comprises bone tissue. In further embodiments, the implantable trap device comprises a porous calcium phosphate ceramic. In other embodiments, the decoy trap comprises a composite vascular structure. In yet further embodiments, the decoy trap further comprises a labeled probe that specifically binds to cancer cells. In other embodiments, the decoy trap is implanted in contact with a blood vessel.

Another aspect of the invention provides a method of diagnosing cancer metastasis in a subject, comprising implanting a decoy trap within the subject, the decoy trap comprising an implantable trap device including a metastatic cancer attractant, allowing the decoy trap to remain within the subject for a period of time, and diagnosing the subject as having metastatic cancer if metastatic cancer cells are detected in the decoy trap. In some embodiments, the method is used to diagnose metastasis in a subject diagnosed with breast, prostate, or lung cancer. In further embodiments, the decoy trap is removed from the subject before detecting bone-seeking cancer cells in the decoy trap. In other embodiments, the decoy trap remains in vivo while detecting bone-seeking cancer cells in the decoy trap.

In some embodiments of the method of diagnosing metastatic cancer, the cancer metastasis is bone metastasis and the metastatic cancer attractant is a bone-seeking cancer attractant. In other embodiments, the bone-seeking cancer attractant is pericytes. In further embodiments, the pericytes are human pericytes. In some embodiments, the implantable trap device comprises bone tissue. In further embodiments, the implantable trap device comprises a porous calcium phosphate ceramic.

In some embodiments of the method of diagnosing metastatic cancer, the decoy trap further comprises a labeled probe that specifically binds to metastatic cancer cells. In further embodiments, the method comprises determining quantifying the amount of metastatic cancer cells that have entered the decoy trap.

### BRIEF DESCRIPTIONS OF THE DRAWINGS

FIG. 1 provides an exploded view of a decoy trap that has been implanted in a subject.

FIGS. 2A and 2B provide images showing BM vascular appearance and micro computed tomography (μCT) assessment of invaded bones. (A) CD31 (Pecam) immunostaining of PDGF-B mutant and Het mice (control) BM, showing no signs of blood vessels gross abnormalities such as collapse and hemorrhages. (B) Gross appearance, two-dimensional CT scanning, and tri-dimensional renderings—volumetric and surface of Het (control) and PDGF-B mutant (Mut) mice humeri. Note the marked osteolysis area in the Het humerus (arrow).

FIGS. 3A-3C provide graphs and images of BLI of injected PDGF-B mutant and control mice (Het and WT): (A) Imaging 15 minutes, 5 and 12 days post-injection, showing increased skeletal invasion (limbs and spine) in WT and Het mice compared to PDGF-B mutant mice and the disappearance of a metastatic signal at 12 days in PDGF-B mutant mice (grey circles). (B) Absence in PDGF-B mutant and persistence in Het mice of the spine signal (white circle) after adrenal gland removal (white arrows) confirming the non-spinal origin of the signal in PDGF5-B mutant spine (arrow in A). (C) Quantification of signal (photon flux and area covered by tumors) showing statistical difference between PDGF-B mutant and Het mice (*=p<0.01). Data are represented as mean±SEM. Representative mice of n=15 (5 per group).

FIGS. 4A and 4B provide images of gross inspection of distant melanoma dissemination: (A) Craniofacial and appendicular invasion by MCC exhibiting a significant reduction in PDGF-B mutant mice. All skulls and scapulae in the mutants were clear of metastases or had only few/small foci, which contrasts sharply with the multiple major/multifocal invasion observed in Het mice. Spines in PDGF-B mutant mice were clear of metastasis in 2/5 mice, or harbored only 1-2 small foci restricted to one vertebral segment in the remaining 3 animals. WT and Het mice had multiple multi-segment metastases in all animals. Both distal femur and proximal tibia were compromised bilaterally in all WT and Het controls, while bilateral invasion was observed in only 1 of 5 PDGF-B mutant mice, with remaining 4 mice exhibiting only one compromised leg that was restricted to the proximal tibia. (B) Melanoma invasion to other target organs with comparable results in both genotypes except for liver (reduced in PDGF-B mutant mice). Arrows: adrenal glands. Circle: adrenal gland agenesis. Three representative mice shown of n=15 (5 per group).

FIGS. 5A-5C provide images showing the histology of metastatic bone tumors. (A, B) Metastatic tumors (T) in distal femur (A) and liver (B) are smaller or absent in PDGF-B mutant mice. Bars=200 μm (low magnification) and 10 μm (high magnification). Melanin-producing B16F10 cells (arrows) localize in the abluminal side of BM sinusoids (Sin), extending to the tissue parenchyma (impaired in PDGF-B mutant mice). (C) CD146 immunohistochemistry (IHC) in BM sections from Het mice. Engrafted B16F10 cells (arrows) physically associate with CD146-positive BM-MSC/pericytes (signal) at the perivascular space and inside the parenchyma (circle). Dotted line=boundary between tumor-invaded and tumor-free BM. Bar=10 μm.

FIGS. 6A-6C provide graphs and images showing induced gene silencing in B16F10 and hMSCs. Effective CD146 silencing (90%) in B16F10 (A) and hMSCs (75%) (B), assessed by immunocytochemistry (ICC) in cultured cells. (C) Sdf-1/CXCL12 effective gene silencing (55%) in hMSCs evidenced by qPCR from RNA obtained from cultured cells. * (p<0.05). Bars=200 μm.

FIGS. 7A-7C provide graphs and images showing the invasion of engineered B16F10 MCC: Reduced invasion of CD146-silenced MCC to craniofacial, appendicular structures and liver (compared to NT-shRNA), evaluated by BLI (A) and gross examination (B & C). BLI signal reached statistical significance (*=p<0.01). Data are represented as mean±SEM. Representative mice of n=6 (3 per group).

FIG. 8 provides a schematic representation of humanized extraskeletal ossicle conformation. The schematic representation of the ossicle showing theoretical cell dispositions within the tissue structure before and after silencing induction with IPTG administration in the drinking water. A timeline of the experiment is also shown. Adip=Adipocyte; Sin=Sinusoid; MK=Megakaryocyte.

FIGS. 9A and 9B provide images showing the human origin of perivascular cells in ossicles and in vivo silencing of CD146. (A) Immunodetection (IHC) of the MSC marker CD271 (blue signal) with a human specific antibody evidences the donor (human) origin of perivascular cells in the engineered ossicles. (B) Immunodetection (IHC) of CD146 in ossicle sections revealing its presence in perivascular cells (arrows) in control structures, significantly reduced in ossicles made with CD146-silenced hMSCs. Bars=200 μm.

FIGS. 10A-10C provide graphs and images showing the B16F10 MCC invasion to humanized extraskeletal ossicles. (A) MCC invasion of the skeleton (top row BLI) and specific implanted ossicles, evaluated by direct examination after harvesting (middle row) and by BLI (bottom row). The signal from the cubes BLI was quantified (photon flux) giving statistical difference of all groups compared to control (*=p<0.01). Data are represented as mean±SEM. Representative mice and cubes of n=8. (B) Histological analysis (H&E staining) of harvested ossicles shows significant melanoma invasion in structures made with control MSC while significantly reduced or absent with Sdf-1/CXCL12 and CD146-silenced cells. SD cubes exhibit no bone and vasculature formation. Bar=200 μm. (C) Immunolocalization (IHC) of CD146 (arrow) in sections from Control ossicles showing invading MCC physically associated with MSC/pericytes at the perivascular space surrounding sinusoids (Sin), and advancing towards the tissue parenchyma as a cell complex (red circle). Bar=10 μm.

FIGS. 11A and 11B provide a cross-sectional representation and images showing the in vitro transendothelial migration (TEM) assay: (A) Schematic representation of the modified TEM. (B—top row) Fluorescence microscopy of DiI-labeled hMSC expressing either non-target (NT) or Sdf-1/CXCL12 silencing vectors (Sdf-1_shRNA) and papillary dermal Fibroblasts seeded at the bottom surface of an 8 μm pore diameter insert membrane. (B—bottom row) Merged bright field and fluorescence microscopy showing B16F10 melanoma cell invasion to the membrane and interaction with seeded cells (Bar=200 μm). Representative pictures from three independent experiments.

FIGS. 12A and 12B provide a schematic representation and image showing the in vitro transendothelial migration assay and a result from the assay. (A) DiI-labeled hMSC (red) expressing either nontarget (NT) or Sdf-1/CXCL12 silencing vectors (Sdf-1_shRNA) seeded at the surface of the transwell at varying distances from the insert. (B) No invasion of MCC to the membrane was observed at all distances (one field shown), confirming the lack of migration by B16F10 cells.

FIGS. 13A and 13B provide a schematic representation of the proposed model. (A) PDGFR-β-expressing MSCs are recruited as pericytes by endothelial cells (EC)-secreted PDGF-B retained in the heparan sulfate-rich pericellular matrix. In their perivascular location, they create an Sdf-1/CXCL12 gradient across the endothelium (black dots) attracting CXCR4-expressing melanoma cells. Sdf-1/CXCL12 is also expressed in smaller quantities by ECs. Cell-cell interactions mediated by homotypic CD146 binding generates that this cellular complex then enters the BM parenchyma. The absence of MSCs in their normal perivascular niche disrupts these mechanisms.

FIGS. 14A and 14B provides image obtained using two-photon microscopy of the habitat (ossicle). (A) Three-dimensional reconstructions of the habitat made with eGFP-hMSCs reveals close proximity of hMSCs to vasculature (arrows). (B) Melanoma (B16F10) were intra-arterially injected and imaged 10 days after injection. Tumor cells can be observed near the vasculature of the habitat with hMSCs now detached from their perivascular locations. Intravital microscopy of the habitat was performed using a Leica SP5 fitted with a DM6000 stage, a 20× water immersion lens, and a 16 W Ti/Sapphire IR laser. Fluorescent dextran was injected into the circulation to illuminate blood vessel prior to imaging.

FIGS. 15A and 15B provide images showing the differential effect of laminins 411 and 511 on cancer cell migration in the presence of hMSCs. Membrane inserts were coated with recombinant laminins 411 and 511 (10 ug/ml). Human MSCs were seeded on the opposite side of each membrane (w/o HUVEC and matrigel). After 24 h, cancer cell lines expressing dTomato fluorescent protein were seeded on the membrane inserts and allowed to migrate for 48 h. Non-migrating cells were scraped from the top of the membrane with a cotton swap. A higher migration rate was observed when laminin 411 was present. The same effect was observed using A375. MDA-231 migration showed no response to laminin 411 or 511. Interestingly, this correlates with the fact that B16F10, PC3 and A375 highly express CD146; however, MDA-231 had the lowest expression.

FIGS. 16A-16D provide images and a graph showing the differential effect of laminins 411 and 511 on cancer cell migration in the presence of hMSCs. Transmigration of B16F10 cells increased in the presence of recombinant laminin 411 and was inhibited in the presence of laminin 511 (A, B). Membrane inserts were coated with recombinant laminins 411 or 511 (10 ug/ml). Human MSCs were seeded on the opposite side of the membrane. After 24 h, melanoma (A375) expressing dTomato were seeded and allowed to migrate for 48 h. Non-migrating cells were scraped from the top of the membrane with a cotton swap. Number of cells that migrated were quantified using Image-J software. (C) hMSC silenced for Lama4 (shLama4) do not support A375 transmigration and when Lama5 is silenced, A375 transmigration is enhanced. These results confirm the permissiveness of laminin 4 for the transmigration of melanoma. (D) provides a graphic representation of a side-view of the transendothelilal migration (TEM) assay.

### DETAILED DESCRIPTION

The methods and techniques described herein are generally performed according to conventional methods well-known in the art and as described in various general and more specific references that are cited and discussed throughout the present specification unless otherwise indicated. See, e.g., Sambrook et al., Molecular Cloning: A Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990).

### Definitions

For clarification in understanding and ease in reference a list of terms used throughout the brief description section and the remainder of the application has been compiled here. Some of the terms are well known throughout the field and are defined here for clarity, while some of the terms are unique to this application and therefore have to be defined for proper understanding of the application.

“A” or “an” means herein one or more than one; at least one. Where the plural form is used herein, it generally includes the singular.

As used herein, the term “diagnosis” can encompass determining the likelihood that a subject will develop a disease, or the existence or nature of disease in a subject. The term diagnosis, as used herein also encompasses determining the severity and probable outcome of disease or episode of disease or prospect of recovery, which is generally referred to as prognosis). “Diagnosis” can also encompass diagnosis in the context of rational therapy, in which the diagnosis guides therapy, including initial selection of therapy, modification of therapy (e.g., adjustment of dose or dosage regimen), and the like.

As used herein, the term “prognosis” refers to a prediction of the probable course and outcome of a disease, or the likelihood of recovery from a disease. Prognosis is distinguished from diagnosis in that it is generally already known that the subject has the disease, although prognosis and diagnosis can be carried out simultaneously. In the case of a prognosis for metastatic cancer, the prognosis categorizes the relative severity of the metastasis, which can be used to guide selection of appropriate therapy for the metastatic cancer.

“Mesenchymal stem cells” or “MSCs” are derived from the embryonal mesoderm and can be isolated from many sources, including adult bone marrow, peripheral blood, fat, placenta, and umbilical blood, among others. MSCs can differentiate into many mesodermal tissues, including muscle, bone, cartilage, fat, and tendon. There is considerable literature on these cells. See, for example, U.S. Pat. Nos. 5,486,389; 5,827,735; 5,811,094; 5,736,396; 5,837,539; 5,837,670; and 5,827,740. See also Pittenger, M. et al, Science, 284:143-147 (1999).

“Stem cell” means an undifferentiated cell that can undergo self-renewal (i.e., progeny with the same multi-differentiation potential) and also produce progeny cells that are committed to a particular differentiation lineage. Within the context of the invention, a stem cell would also encompass a more differentiated cell that has de-differentiated, for example, by nuclear transfer, by fusion with a more primitive stem cell, by introduction of specific transcription factors, or by culture under specific conditions. See, for example, Wilmut et al., Nature, 385:810-813 (1997); Ying et al., Nature, 416:545-548 (2002); Guan et al., Nature, 440:1199-1203 (2006); Takahashi et al., Cell, 126:663-676 (2006); Okita et al., Nature, 448:313-317 (2007); and Takahashi et al., Cell, 131:861-872 (2007).

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Prevention or prophylaxis, as used herein, refers to preventing the disease or a symptom of a disease from occurring in a subject which may be predisposed to the disease but has not yet been diagnosed as having it (e.g., including diseases that may be associated with or caused by a primary disease). Prevention may include completely or partially preventing a disease or symptom. With regard to cancer metastasis, prevention refers to avoiding or decreasing the number of secondary cancer sites that form outside of the primary tumor.

The terms “therapeutically effective” and “pharmacologically effective” are intended to qualify the amount of an agent which will achieve the goal of improvement in disease severity and the frequency of incidence over treatment of each agent by itself, while avoiding adverse side effects typically associated with alternative therapies. The effectiveness of treatment may be measured by evaluating a reduction in tumor load or decrease in tumor growth in a subject in response to the administration of anticancer agents. The reduction in tumor load may be represent a direct decrease in mass, or it may be measured in terms of tumor growth delay, which is calculated by subtracting the average time for control tumors to grow over to a certain volume from the time required for treated tumors to grow to the same volume. In the case of antimetastatic agents, the effectiveness of treatment may be measured by evaluating whether treatment has prevented or decreased the spread of the cancer from the original source to new tissues.

The terms “individual,” “subject,” and “patient” can be used interchangeably herein irrespective of whether the subject has or is currently undergoing any form of treatment. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). Treatment or diagnosis of humans is of particular interest.

### Treating Cancer Metastasis

In one aspect, the present invention provides a method of treating or preventing cancer metastasis in a subject, comprising implanting a decoy trap within the subject, the decoy trap comprising an implantable trap device including a metastatic cancer attractant. The decoy trap catches metastasizing cancer cells by drawing them into the decoy trap as a result of attraction of the metastasizing cancer cells to the implantable trap device and/or the metastatic cancer attractant, thereby preventing or decreasing the number of circulating cancer cancer cells that can become established as metastatic cancer in other tissue of the subject.

Cancer is a disease of abnormal and excessive cell proliferation. Cancer is generally initiated by an environmental insult, certain gene mutations or gene deletions, or error in replication that allows a small fraction of cells to escape the normal controls on proliferation and increase their number. The damage or error generally affects the DNA encoding cell cycle checkpoint controls, or related aspects of cell growth control such as tumor suppressor genes. As this fraction of cells proliferates, additional genetic variants may be generated, and if they provide growth advantages, will be selected in an evolutionary fashion. Cells that have developed growth advantages but have not yet become fully cancerous are referred to as precancerous cells. Cancer results in an increased number of malignant cells in a subject. These cells may form an abnormal mass of cells called a tumor, the cells of which are referred to as tumor cells. The overall amount of tumor cells in the body of a subject is referred to as the tumor load. Tumors can be either benign or malignant. A benign tumor contains cells that are proliferating but remain at a specific site and are often encapsulated. The cells of a malignant tumor, on the other hand, can invade and destroy nearby tissue and spread to other parts of the body through a process referred to as metastasis.

As used herein, “metastasis” refers to the ability of cells of a cancer (e.g. a primary tumor, or a metastatic tumor) to be transmitted to other locations in the subject (i.e., target organs) and to establish new tumors at such locations. The metastatic process involves a number of different steps. After creation of the initial tumor mass, the tumor is vascularized with a capillary network from the surrounding host tissue. Intravasation of the capillary network by tumor cells then occurs, resulting in the creation of circulating cancer cells. Thin-wall venule-like lymphatic channels offer very little resistance to penetration by tumor cells and provide the most common pathways for tumor cells entry into the circulation, although in some cases detachment and embolization of small tumor cell aggregates occur. Tumor cells that survive circulation may then arrest in the capillary beds of organs, after which extravasation occurs, through interaction of the circulating tumor cell with the endothelium, and facilitated by pericytes. Proliferation within the organ parenchyma completes the metastatic process.

The invention is used to treat or prevent cancer metastasis. The most common places for the metastases to begin are referred to as the primary cancer, and include are the lung, breast, skin, colon, kidney, prostate, pancrease, liver, and cervix. There is a propensity for certain tumors to seed in particular organs. The propensity for a metastatic cell to spread to a particular organ is termed ‘organotropism’. For example, prostate cancer usually metastasizes to the bones. In a similar manner, colon cancer has a tendency to metastasize to the liver. Stomach cancer often metastasises to the ovary in women. In some embodiments, the method is used to treat the metastasis originating from breast cancer, prostate cancer, or lung cancer primary tumors. The cells capable of forming metastatic cancer are circulating cancer cells are those that move within the bloodstream, as opposed to cancer cells present at a fixed location, such as a solid tumor. The circulating cancer cells form metastatic cancer cites by transendothelial migration.

A circulating tumor cell, as used herein, is a cell that can circulate through blood vessels but is capable of forming a new cancer site upon extravasation. A variety of types of circulating tumor cells can be used in a method of the invention, including cells from metastatic epithelial cancers, carcinomas, melanoma, leukemia, etc. The tumor cells may be, e.g., from cancers of breast, lung, colon, bladder, prostate, liver, gastrointestinal tract, endometrium, tracheal-bronchial tract, pancreas, liver, uterus, ovary, nasopharynges, prostate, bone or bone marrow, brain, skin or other suitable tissues or organs. In a preferred embodiment, the cancer cells are of human origin.

Transendothelial migration refers to the migration of cells through the various layers making up the blood vessel, and in particular the endothelial layer. Transendothelial migration includes extravasation and intravasation. Extravasation refers to migration of a cell (e.g., a circulating tumor cell) from within the blood vessel to outside of the blood vessel (e.g, to an organ or tissue such as bone), while intravasation refers to migration of a cell from outside the blood vessel (e.g., at the site of a primary tumor) to within the blood vessel.

Pericytes can play an active role in facilitating transendothelial migration through the endothelium of the blood vessel. Facilitation works via the creation of both cancer cell-attracting molecular gradients and docking points for cell-cell attachment (both exerted by the facilitators). These mechanisms attract cancer cells close to the endothelium capturing them with further migration outside the blood vessel. For example, carbohydrate-mediated cancer cell adhesion to the vascular endothelium is involved in the metastasis of a wide variety of cancers, including gastric, colorectal, pancreatic, liver, ovary, head and neck, and breast cancers, etc. In some embodiments, blood vessel transendothelial migration is facilitated extravasation, while in other embodiments blood vessel transendothelial migration is facilitated intravasation. In further embodiments, the blood vessel transendothelial migration is the facilitated extravasation of circulating cancer cells (e.g., melanoma cells) into the tissue model (e.g., bone tissue) of the invention.

Pericytes are contractile cells that wrap around the endothelial cells of blood vessels throughout the body. Also known as Rouget cells or mural cells, pericytes are embedded in basement membrane where they communicate with endothelial cells of the blood vessels by means of both direct physical contact and paracrine signaling. Pericytes regulate capillary blood flow, the clearance and phagocytosis of cellular debris, and the permeability of the blood vessel to other cells, including circulating cancer cells. A variety of integrin molecules and other factors are involved in communication between pericytes and endothelial cells separated by the basement membrane.

The decoy traps of the invention can be used to treat or diagnose the metastasis that is directed to various secondary tissues, based on the organotropism of the particular metastatic cancer and the ability of the decoy trap to retain circulating cancer cells that have contacted the decoy trap. In some embodiments, the method is used to treat or prevent the development of bone metastasis. Bone-originating primary tumors such as osteosarcoma, chondrosarcoma, and Ewing's sarcoma are rare, and bone cancer can occur as a result of metastasis to the bone. While many types of cancer are capable of forming metastatic tumors within bone, the microenvironment of the marrow tends to favor particular types of cancer, including prostate, breast, and lung cancers. Particularly in prostate cancer, bone metastases tend to be the only site of metastasis. The most common sites of bone metastases are the spine, pelvis, ribs, skull, and proximal femur. Although the prevalence of melanoma bone metastasis is lower than these cancers, the mortality rate of bone metastasis due to melanoma is the highest among these cancers. Symptoms of bone metastases severe pain, bone fractures, spinal cord compression, hypercalcemia, anemia, spinal instability, and decreased mobility. The presence of bone metastases can be confirmed using a CT scan.

The present invention employs a decoy trap—also referred to as an implanted tissue mimicking model—for treating or diagnosing cancer metastasis. The decoy trap comprises an implantable trap device including a metastatic cancer attractant. The implantable trap device is a three-dimensional tissue model reflective of a biological tissue, particularly an organ such as bone or liver, to which cancer cells might be expected to metastasize based on its organotropic predilection. An organ or tissue, as used herein, refers to a differentiated part of an organism which has a specific function. In some embodiments, the implantable trap device is composed of normal animal tissue obtained from a suitable animal subject, or a different compatible subject. In other embodiments, the implantable trap device is an artificial tissue model such as an organoid, or that has been otherwise engineered using cell culture or inorganic materials. Tumor cells are retained within the implantable trap device by molecular specific attachment to the tissue. For example, in the case of bone tissue, the tumor cells are pulled into the stroma of bone marrow and become immobilized by their attachment to the bone stroma.

The form of the implantable trap device is not limited, and may be, for example, an epithelial tissue device that can be constructed by culturing, for example, surface epithelial cells and glandular epithelial cells; a connective tissue device that can be constructed by culturing, for example, fibroblasts and fat cells; a muscle tissue device that can be constructed by culturing, for example, myoblasts, cardiac muscle cells, and smooth muscle cells; a nerve tissue device that can be constructed by culturing, for example, nerve cells and glial cells; and an organoid model that can be constructed from a combination of cells derived from two or more tissues. The cells used are not limited to normal mature differentiated cells, and may be undifferentiated cells such as embryonic stem cells, somatic stem cells, and induced pluripotent stem cells; focus-derived cells such as cancer cells; or transformants transfected by exogeneous genes. Preferably the implantable trap device is formed of tissue for which the target metastatic cancer has an organotropic affinity.

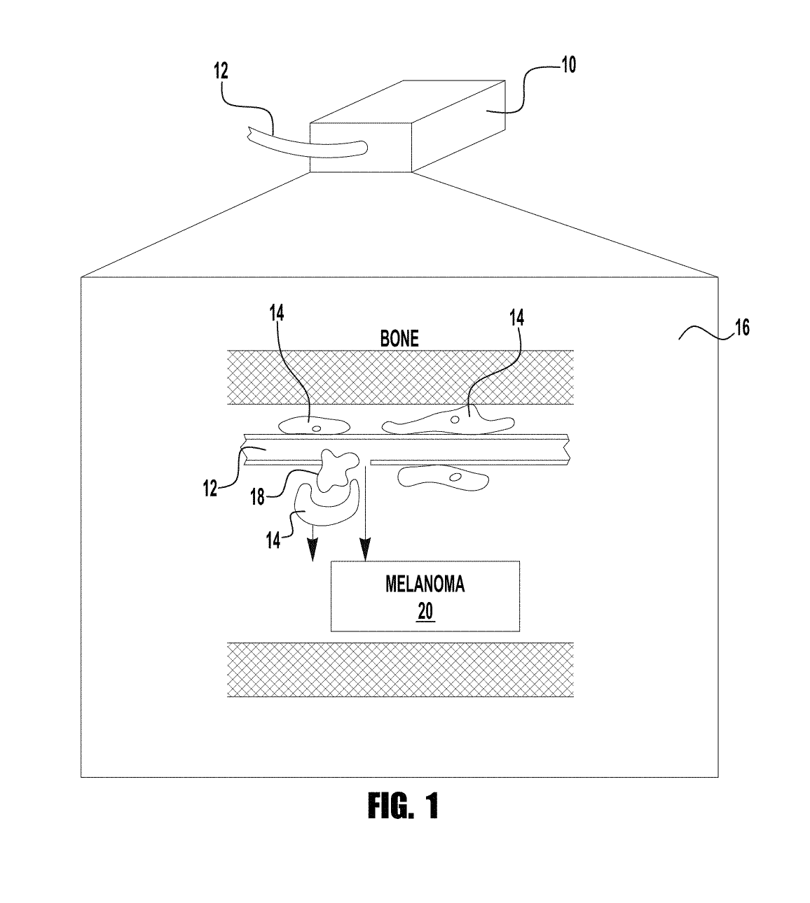

One embodiment of the implantable trap device 10 is shown in FIG. 1. The implantable trap device 10 is positioned around a blood vessel 12. For example, the implantable trap device 10 can be implanted in a cancer patient in the same manner used to implant drug ports. The expanded view shows the process of removing cancer (e.g., melanoma) cells within the implantable trap device 10. Positioned within the implantable trap device 10 and around the blood vessel 12 are a plurality of metastatic cancer attractants 14 (e.g., pericytes). The implantable trap device 10 is made up of a decoy matrix material 16. Lining the blood vessel is the basement membrane 18, or in some cases basal lamina, which is made by both the vascular endothelium and perivascular cells. Metastatic cancer cells 20 (e.g., melanoma) enter the implantable trap device 10 through the blood vessel, and are attracted into the decoy matrix material 16 by the metastatic cancer attractant 14, after which they are trapped within the decoy matrix material 16 of the device.

In some embodiments, the implantable test device is a bone tissue device. Bone tissue devices can be made of organic or inorganic scaffold material, and mimic bone tissue, typically for bone tissue engineering purposes. A variety of bone tissue models are known to those skilled in the art. See Sarkar, S, Lee, B, Korean J Intern Med. 2015, 30(3):279-293. For example, bone tissue devices can be constructed using bioceramics, glass and glass ceramics, biopolymers, and graphene. In particular, porous ceramic materials are useful as bone tissue models. Dennis J., Caplan A., J Oral Implantol. 1993; 19(2):106-15. In some embodiments, the implantable trap device comprises a porous calcium phosphate, such as a porous calcium phosphate ceramic cube. Samavedi et al., Acta Biomater. 2013 9(9):8037-45. In further embodiments, the porous calcium phosphate is embedded in a natural or extruded polymer. For example, in some embodiments, the porous calcium phosphate is embedded in the natural polymer collagen coated with hydroxylapatite.

The decoy trap is implanted into the subject in a manner which allows or encourages acceptance by the subject. For example, in some embodiments, the decoy trap may be implanted within a subject using an implantation guide or guide wire configured to be visible using ultrasound, allowing the implantation procedure to be guided using ultrasound imaging. In some embodiments, a single decoy trap is implanted into the subject, while in other embodiments a plurality of decoy traps are implanted into the subject. Use of a plurality of decoy traps in a subject can be useful for the treatment or diagnosis of different types of metastatic cancer, to increase effectiveness by placement in various spatially discrete organs or tissue, or for other purposes.

In some embodiments, the decoy trap is implanted in contact with a blood vessel. Blood vessels are generally cylindrical conduits that are part of the circulatory system that transports blood throughout the body. An arrangement of blood vessels is referred to as vasculature. Blood vessels include arteries, capillaries, and veins. The space within the blood vessel is referred to as the lumen, while the first layer adjacent to the lumen is the endothelium. The endothelium is a single layer of simple squamous endothelial cells glued by a polysaccharide intercellular matrix. Outside of the endothelium is the basement membrane, which is a thin, fibrous, non-cellular region of tissue that separates the endothelium from the surrounding smooth muscle and connective tissue. Capillaries have a significantly simpler structure, consisting of endothelium surrounded by a basement membrane. Capillaries can be fenestrated (i.e., equipped with diaphragms that regulate the size of the fenestration) or non-fenestrated (i.e., open pore capillaries or sinudoids).

In addition to an implantable trap device, the decoy trap also includes a metastatic cancer attractant. The metastatic cancer attractant should serve to reinforce the ability of the implantable trap device to attract metastatic cancer having an organotropic affinity for the type of tissue being modeled by the implantable trap device, and maintain the cancer cells within the trap once they have been attracted. Examples of cancer attractants are the cellular components (e.g., MSC, pericytes) and other perivascular niche components and molecules such as CXCL12 (chemochine) and laminins 411 and 421, which are components of the vascular basement membrane. In some embodiments, the decoy trap is configured to attract metastatic bone cancer cells, and the metastatic cancer attractant is a bone-seeking cancer attractant. An example of a bone-seeking cancer attractant is a pericyte.

Other cancer attractants include compounds known to encourage transendothelial migration. For example, the compound may be known to have an effect on the adhesion of cancer cells to blood vessels. In additional embodiments, the agent is known to have an affect on the activity of pericytes. Examples of agents that have an effect on the activity of a pericyte-affecting molecule can be selected from the group consisting of a platelet-derived growth factor (PDGF)-BB/PDGF receptor β, a membrane type 1-matrix metalloproteinase, heparin sulphate proteoglycans, N-cadherin, Ang1/Tie-2, transforming growth factor β, hepatocyte growth factor, ephrinB2, vascular cell adhesion molecule 1/α4-integrin, CD146, Sdf-1/CXCL12, fibronectin, and laminin (e.g., laminin 411 and laminin 421). Additional factors known to attract circulating cancer cells include various peptides and carbohydrates involved in cell signaling and adhesion, such as CD146. Factors that can be identified involve constitutive and induced molecules expressed in either the invading cancer cell or the local/resident pericyte. These include secreted growth factors and their cognate receptors, cell-cell adhesion molecules and cell-ECM interacting molecules.

In some embodiments, the decoy trap initially comprises mesenchymal stem cells. Mesenchymal stem cells are characterized by their ability to produce and secrete structural molecules commonly found within the extra cellular matrix (ECM), though it is well established that mesenchymal stem cells also produces an array of different signaling molecules which can function in both the differentiation of cells (as in the case of the developing organ) as well as the maintenance of stemness, as seen in the mesenchymal components of the intestinal stem-cell niche. The expression patterns of mesenchymal stem cells have been shown to be vastly different dependent on the site of origin, though some general markers expressed in multiple mesenchymal lineages are vimentin, fibronectin and various forms of collagen. In contrast to the cells of the epithelial components of a developing organ mesenchyme is marked by a migrating capability.

Mesenchymal stem cells reside as pericytes in the abluminal aspect of blood vessels (i.e., perivascular niche) in all vascularized tissues in the body. Mesenchymal stem cells have the capacity of recognizing sites of injury, where they reassemble as pericytes and exert immunomodulatory and trophic activities locally. In some embodiments, the subject is human and the pericytes are human pericytes.

Upon implantation into the subject, a proportion of the loaded mesenchymal stem cells into the tissue implantable trap device differentiate into ECM-producing cells (e.g., osteoblasts in bone), while some migrate to the basement membrane of the nascent blood vessels that form around and inside the implanted tissue mimicking model, readopting their perivascular nature (i.e., as pericytes). Accordingly, in some embodiments, the decoy trap comprises a composite vascular structure. The system of blood vessels, including pericytes, that forms around and inside the decoy trap is referred to herein as a composite vascular structure, which serves to provide vascular support for the decoy trap. Note that while differentiation of the mesencymal cells typically occurs subsequent to implantation, that differentiation of the mesenchymal cells can also be carried out ex vivo in some embodiments of the invention, before implantation.

The decoy traps can prevent the circulating cancer cells from forming metastatic cancer by trapping and isolating them, thereby inhibiting metastatic progression. For example, the decoy traps may result in the delayed appearance of secondary tumors, slowed development of primary or secondary tumors, decreased occurrence of secondary tumors, slowed or decreased severity of secondary effects of disease, among others. In the extreme, complete inhibition is achieved, and is referred to herein as prevention (e.g., virtually complete inhibition, no metastasis if it had not occurred, no further metastasis if there had already been metastasis of a cancer, or virtually complete inhibition of the growth of a primary tumor caused by re-seeding of the tumor by a circulating cancer cell. In some embodiments, the decoy traps are removed from the subject after a period of time, or upon detection of metastatic cancer cells within the decoy traps, in order to permanently remove the metastatic cancer cells from the subject.

In some embodiments, the decoy trap can be combined with other forms of treatment for cancer (e.g., metastatic cancer). Additional methods of anticancer therapy include one or more methods selected from the group consisting of surgery, cryoablation, thermal ablation, radiotherapy (e.g., external beam radiotherapy), chemotherapy, radiofrequency ablation, electroporation, alcohol ablation, high intensity focused ultrasound, photodynamic therapy, administration of monoclonal antibodies, and administration of immunotoxins. Examples of agents suitable for use in anticancer therapy include cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (paclitaxel, docetaxel), cytochalasin B, gramicidin D, ethidium bromide, emetine, mitomycin, etoposide, tenoposide, vincristine, vinblastine, vinorelbine, colchicin, anthracyclines (doxorubicin and epirubicin) daunorubicin, dihydroxy anthracin dione, mitoxantrone, mithramycin, actinomycin D, adriamycin, 1-dehydrotestosterone, glucocorticoid, procaine, tetracaine, lidocaine, propranolol, puromycin, ricin, and maytansinoids, Gefitinib, Erlotinib, Lapatinib, Sorafenib, Sunitinib, Imatinib, Dasatinib, Nilotinib, temsirolimus, everolimus, rapamycin, Trastuzumab, Cetuximab, Panitumumab, Bevacizumab, Rituximab, and Tositumomab.

### Methods of Diagnosing Cancer Metastasis in a Subject

In another aspect, the invention provides a method of diagnosing cancer metastasis in a subject. The method includes implanting a decoy trap within the subject, the decoy trap comprising an implantable trap device including a metastatic cancer attractant, allowing the decoy trap to remain within the subject for a period of time, and diagnosing the subject as having metastatic cancer if metastatic cancer cells are detected in the decoy trap. In some embodiments, the decoy trap remains in vivo while detecting bone-seeking cancer cells in the decoy trap. In other embodiments, the decoy trap is removed from the subject before detecting bone-seeking cancer cells in the decoy trap (i.e., the metastatic cancer cells are detected ex vivo).

The metastatic cancer being diagnosed can be any of the types of metastatic cancer described herein. In addition, the method can be used to diagnose metastasis in a subject who has already been diagnosed with cancer, and in particular cancers known to commonly lead to metastasis such as breast, prostate, or lung cancer. Likewise, the decoy trap used in the method of diagnosis can include any of the features described herein. For example, in some embodiments, the cancer metastasis is bone metastasis and the metastatic cancer attractant is a bone-seeking cancer attractant. In further embodiments, the bone-seeking cancer attractant is pericytes, such as human pericytes. In other embodiments, the implantable trap device comprises bone tissue. In further embodiments, the implantable trap device comprises a porous calcium phosphate ceramic.

In some embodiments, the decoy trap further comprises a labeled probe that specifically binds to metastatic cancer cells. These probes can be used to detect the presence of metastatic cancer cells in the decoy trap either in methods of treatment or methods of diagnosis. Any of a variety of conventional labels can be used to label metastatic cancer cells. For example, suitable labels include green fluorescent protein (GFP), red fluorescent protein (RFP), and luciferase, whose use is described in the Examples. Other conventional labels include DsRed, EYFP, ECFP, EVFP and derivatives of EGFP. See also the markers listed at the web site of BD Biosciences (Clontech). When it is desirable to label two different cell populations at the same time (e.g., metastatic cancer cells and cells of a resident tumor, or metastatic cancer cells inoculated at two different (e.g. orthotopic and/or ectopic) sites in a subject), labels which can be easily distinguished can be used. For example, a first cell type can be labeled with a GFP and a second cell type with RPF; or a first cell type can be labeled with firefly luciferase and a second cell type with Renilla luciferase.

Methods of detecting (e.g., quantitating) detectably labeled metastatic cancer cells in the decoy trap will be evident to the skilled worker. For example, when a metastatic cancer cell is labeled with a fluorescent marker, it can be detected by examining, with a fluorescent microscope, a tissue sample from the tumor of the subject. When a metastatic cancer cell is labeled with luciferase, the tumor can be examined in the living subject (e.g. in real time) by measuring light emission (bioluminescence) from the marker.

Methods of detection can be readily quantified by non-invasive photon flux emission measurement of luminescence (luciferase), non-invasive imaging of fluorescence, ex-vivo imaging of luminescence, ex-vivo imaging of fluorescence, fluorescence-activated sorting of tumor cells after dissociation of the extracted tumors into a cell suspension, immunohistochemical analysis of marker proteins, to provide quatitative, reproducible assays. For example, in some embodiments, the method further comprises determining quantifying the amount of metastatic cancer cells that have entered the decoy trap.

In some embodiments, the metastatic cancer cells trapped within the decoy trap are characterized to determine the type of metastatic cancer cells or to characterize the metastatic cancer cells in other ways. For example, the metastatic cancer cells can be genetically analyzed to determine their genetic characteristics using methods such as PCR analysis. In further embodiments, the metastatic cancer cells trapped within the decoy trap are removed and implanted in another setting, such as cell culture or an immunocompromized animal model, in order to further characterize the metastatic cancer cells or determine their susceptibility to differing types of chemotherapeutic agents.

The following examples are included to demonstrate a preferred embodiment of the invention. It should be appreciated by those of skill in the art that the techniques disclosed in the example, which follow represent techniques discovered by the inventor to function well in the practice of the invention, and thus can be considered to constitute a preferred mode for its practice. However, those of skill in the art should, in light of the present disclosure, appreciate that many changes can be made in the specific embodiments which are disclosed and still obtain a like or similar result without departing from the spirit and scope of the invention.

### Examples

### Example 1: Mesenchymal Stem Cells Regulate Melanoma Cancer Cells Extravasation to Bone and Liver at their Perivascular Niche

Skeleton and liver are preferred organs for cancer dissemination in metastatic melanoma negatively impacting quality of life, therapeutic success and overall survival rates. At the target organ, the local microenvironment and cell-to-cell interactions between invading and resident stromal cells constitute critical components during the establishment and progression of metastasis. Mesenchymal Stem Cells (MSCs) possess, in addition to their cell progenitor function, a secretory capacity based on cooperativity with other cell types in injury sites including primary tumors (PT). However, their role at the target organ microenvironment during cancer dissemination is not known. We report that local MSCs, acting aspericytes, regulate the extravasation of melanoma cancer cells (MCC) specifically to murine bone marrow (BM) and liver. Intra-arterially injected wild-type MCC fail to invade those selective organs in a genetic model of perturbed pericyte coverage of the vasculature (PDGF-B^{ret/ret}), similar to CD146-deficient MCC injected into wild type mice. Invading MCC interact with resident MSCs/pericytes at the perivascular space through co-expressed CD146 and Sdf-1/CXCL12-CXCR4 signaling. Implanted engineered bone structures with MSCs/pericytes deficient of either Sdf-1/CXCL12 or CD146 become resistant to invasion by circulating MCC. Collectively, the presence of MSCs/pericytes surrounding the target organ vasculature is required for efficient melanoma metastasis to BM and liver.

### Materials and Methods

PDGF-B^{ret/ret} mice:

PDGF-B^{ret/ret} transgenic mice (PDGF-B mutant) were kindly provided by Drs. Betsholtz and Genové (Karolinska Institute, Stockholm, Sweden). These transgenic mice (C57B1/6 background) express a mutant PDGF-B that lacks a C-terminal retention motif required to confine this growth factor to the EC compartment, necessary for the recruitment of pericyte progenitors expressing PDGFRB. Armulik et al., Circ Res 2005; 97(6), 512-523. The impaired PDGF-B binding results in defective pericyte recruitment and coverage of microvessels with fewer pericytes and their partial abluminal detachment from the vessel wall. Gerhardt H, Semb H., J Mol Med 2008; 86(2), 135-144. Given that the Pdgfb^{ret} allele is hypo-functional and PDGF-B^{ret/+} mice are indistinguishable from PDGF-B^{+/+} mice, adult (10 week old) Het, WT and PDGF-B mutant littermate mice (n=5 per group) were used in all experiments. Lindblom et al., Genes Dev 2003; 17(15), 1835-1840.

Bioluminescence Imaging (BLI):

BLI was performed after subcutaneous injection of 200 μl of 12.5 mg/ml of luciferin substrate (Biosynth, Cat# L-8220) using a Xenogen IVIS 200 series system. Fifteen minutes after B16F10 cell infusion, an early BLI was performed to evaluate cell distribution throughout the body. Later, images at days 3, 7 and 12 were obtained to evaluate cancer cells engraftment and their temporal progression as growing metastases. To quantify tumor invasion to target organs, BLI signal was analyzed (d12) in terms of photon flux (photons/second/cm²/steradian) and the area covered by signal (cm²e⁻¹) taken at specific locations (extremities and spine after adrenal glands removal) using a pre-defined geometrical shape.

Gene Silencing in B16F10 MCC and hMSC Cells:

CD146 was silenced in B16F10 MCC using a validated shRNA murine sequence cloned in a regular pLKO.1-puro vector, bacterially amplified, sequence verified and delivered as lentiviral transduction particles ready to use (MISSION® RNAi clone ID: NM_023062.1-656s1c1, Sigma Aldrich, St Louis, Mo.). CD146 and Sdf-1/CXCL12 were silenced in BM-derived hMSC using validated human shRNA sequences cloned into an inducible pLKO-puro-IPTG-3xLacO vector [Isopropyl β-D-1-thiogalactopyranoside (IPTG)-dependent transcriptional induction], also delivered as viral particles (MISSION® RNAi clone IDs: CD146: NM_006500.1-1322s1c1; Sdf-1/CXCL12: NM_000609.4-157s21c1, Sigma Aldrich, St Louis, Mo.). The use of an inducible system is intended to avoid the effects of silencing CD146 and Sfd-1/CXCL12 during the formation of both bone and the sinusoidal network inside the ossicles. For the TEM assay, Sdf-1/CXCL12 gene silencing was induced 5 days before the assay.

In Vitro Transendothelial Migration Assay (TEM):

A modified Boyden chamber cell migration assay was used to quantitate the invasion potential of B16F10 cancer cells in 2 different conditions, relative to pre-labeled hMSCs with the cationic lipophilic dye Dil for their fluorescence detection: A) When the MSC/pericytes are in close contact with the membrane but silenced for Sdf-1/CXCL12; and B) When the distance between the membrane (acting as an endothelium) and the MSC/pericytes is increased, reminiscent of the PDGF-B^{ret/ret} mutant mice “anatomic” phenotype (in vitro counterpart).

Humanized Heterotopic Bone Formation Assay:

A total of 4.5×10⁶ non-transduced hMSC and hMSC expressing inducible vectors for non-target (NT), CD146 (CD146.shRNA) and Sdf-1/CXCL12 (Sdf-1.shRNA) were vacuum-loaded into sterile porous ceramic cube carriers (hydroxyapatite/tricalcium phosphate 40/60—Zimmer, Warsaw, Ind.) pre-coated with a 100 μg/ml solution of fibronectin. Dennis et al., Biomaterials 1998; 19(15), 1323-1328. The cubes were subcutaneously implanted into immunocompromised mice (CB17-Prkdc SCID) for 8 weeks to form extraskeletal bone structures (ossicles). Every animal (n=8) received 4 cubes, each representing one of the conditions tested. In order to minimize any potential anatomical effect, their relative positions were changed in each animal. After 8 weeks, gene silencing at the protein level in hMSC was accomplished by administering IPTG (12.5 mM) in the drinking water for 7 days. At this time, 1×10⁶ murine B16F10 MCC were intra-arterially injected. BLI images at days 1, 3, 7 and 12 were taken to evaluate MCC engraftment and growth. Two weeks after cell injections, the animals were sacrificed, imaged and the implanted ossicles analyzed by histology.

Modified B16F10 MCC:

B16F10 cells typically colonize target organs within 2 weeks after systemic injection, at which time metastases can be macroscopically observed as black melanin deposits produced by the engrafted cells. B16F10 cells were lentiviral-transduced with a dual-fusion reporter (fluc-mrfp) encoding firefly luciferase and monomeric red fluorescent protein allowing tracking by Bioluminescence (BLI) and Fluorescence imaging respectively. Love et al., J Nucl Med 2007; 48(12), 2011-2020; Lin et al., Translational Medicine 2012; 1(12), 886-897. Stably transduced cells were sorted by FACS based on red fluorescence intensity, and cells with highest signal collected and serially expanded.

Intra-Arterial Injections of Cancer Cells:

All animal experiments were approved by the local IACUC (Case Western Reserve University) and conform to the U.S. Public Health Service Policy on Humane Care and Use of Laboratory Animals (NIH, Department of Health and Human Services). Five×10⁵ B16F10 cells (in 200 μl of sterile PBS) were delivered intra-arterially under general inhaled anesthesia (2% isoflurane) via a carotid catheter advanced towards the aortic arch, thereby bypassing the lungs and avoiding the first passage effect. Lin et al., Molecular therapy 2013; 22 (1), 160-168. All animals were injected at the same rate (over 2 minutes), monitored for breathing rate and rectal temperature and followed until full recovery from anesthesia.

Clinical Assessment:

General evaluation of mice throughout the experiments includes: body weight, general aspect, degree of mobility and ambulation in an open space, and qualitative assessment of bone cancer-related pain by evaluating movement-evoked limb lifting during walking. Vermeirsch et al., Pharmacol Biochem Behav 2004; 79(2), 243-251. The limb lifting assessment, although subjective, can provide information regarding early functional consequences in colonized bones that can be correlated with the degree of cancer cells engraftment evaluated by BLI, as pain appears before any radiological evidence of bone colonization.

Animal Dissections:

A thorough body dissection was performed in order to directly assess invasion by cancer cells. Given that B16F10 melanoma cells actively produce melanin, the degree of tissue colonization can be initially estimated by the presence of deposited pigment.

Micro-Computed Bone Scans (μCT) and Bone Morphometry:

Bone osteolysis was assessed in paraffin fixed whole bone samples by μCT imaging using a GE Healthcare eXplore Locus machine. Multiple micro-tomographic slices obtained with a resolution of 20 μm were then reconstructed in 3D renderings for further analysis. Morphometric parameters [bone volume/total volume (BV/TV), trabecular number (TbN), trabecular thickness (TbTh), trabecular spacing (TbSp), bone mineral content (BMC) and bone mineral density (BMD)] were calculated in processed samples and used as indicators of an underlying bone phenotype.

Histology and Immunohistochemistry:

For histology (H&E staining) and IHC, harvested bones and ossicles were fixed in 4% Paraformaldehyde (PFA) for 48 h, decalcified in a solution of 12.5% EDTA/2.5% PFA-pH 7.5 for 10 days (4° C.), and then paraffin embedded and sectioned (10 μm). IHC in sections was performed after antigen retrieval (Proteinase K at 37° C.), endogenous peroxidase quenching (3% H₂O₂) and primary antibody incubation using the ImmPRESS™ polymerized reporter enzyme staining system (peroxidase micropolymers) with different enzyme substrates (chromogens) allowing multiple labeling (Vector Labs). The following optimized primary antibodies were used: Rabbit monoclonal anti-CD146 (Millipore, Billerica, Mass., Cat #04-1147); Mouse monoclonal anti-human CD271 (BD Pharmingen Cat #557194) to specifically identify MSC/pericytes of human origin; Rat monoclonal anti-CD31 (Abcam ab56299, Cambridge, Mass.) to identify endothelial cells.

Gene Silencing in B16F10 MCC and hMSC Cells:

Cultures of hMSC were established as previously described. Haynesworth et al., Bone 1992; 13(1), 81-88. The BM was collected using a procedure reviewed and approved by the University Hospitals of Cleveland Institutional Review Board. Cells were obtained from healthy de-identified adult volunteer donors after signing an informed consent. The use of an inducible system is based on the lactose operator-repressor system that efficiently suppresses target gene expression both in vitro and in vivo after 24 h of IPTG administration to mice in the drinking water (12.5 mM). Wu et al., DNA Cell Biol 1997; 16(1), 17-22. In all cases, 100,000 cells per well (6 well plate) were transduced in 1.5 ml total volume containing Protamine Sulfate (100 μg/mL) as coadjuvant, and viral particles at MOI 5. Selection was performed for 10 days with Puromycin (5 μg/ml) with non-transduced cells serving as selection control. Amplified transduced cells were tested for silencing efficiency by immunocytochemistry in coverslip-cultured cells (CD146) and qPCR in regular cultures (Sdf-1/CXCL12) using a non-target (NT) sequence as control. For the inducible silencing vectors, transgene activation was tested using two different concentrations (200 μM and 1 mM) of IPTG (Life technologies, Carlsbad, Calif.) for 6 days. For qPCR assessments, cells were collected by centrifugation for 5 mM at 1200 rpm (110×g) and RNA isolated with TRIzol (Invitrogen) followed by DNaseI digestion and purification with the RNeasy mini kit (Qiagen). One μg of high quality total RNA was retrotranscribed with SuperScript III (Invitrogen), and 10 ng of the resulting cDNA was amplified by qPCR in a StepOne Real-time thermocycler (Applied Biosystems) using SYBR-green. Results werenormalized to the endogenous expression of GAPDH and the fold expression calculated with the 2^{-ΔΔ}CT method.

In Vitro Transendothelial Migration Assay (TEM):

A) When the MSC/pericytes are silenced for Sdf-1/CXCL12: DiI-labeled hMSCs (5×10⁵) expressing either NT_shRNA or Sdf-1/CXCL12_shRNA vectors were treated with IPTG (200 μM) for 5 days to induce gene silencing. They were then cultured at the bottom of an 8 μm pore size polyethylene therephthalate membrane pre-coated with Gelatin (1% for 1 h at 37° C.) and a thin layer of Matrigel (BD BioCoat™ Matrigel™) facing the upper chamber following manufacturer's instructions. As controls, no cells and human papillary dermal fibroblasts were cultured similarly. Forty-eight hours after culturing the coated membrane with the engineered hMSCs, 1×10⁵ B16F10 melanoma cancer cells were seeded in the upper chamber and allowed to migrate through the membrane for additional 48 hours. The upper chamber was then scrapped with a cotton swab to remove unattached cells and the membrane removed from the insert with a scalpel, fixed in 10% Neutral Buffered Formalin and analyzed by bright field and fluorescence microscopy merging both images. B) When the distance between the endothelium and the MSC/pericytes is increased: The TEM was performed comparing the results obtained in A (distance of ˜50 μm) with different distances between the hMSCs and the coated membrane (<1 mm, 1 mm and >1 mm). The hMSCs were plated on the bottom surface of the receiving plate and the B16F10 cancer cells were seeded on the upper chamber coated with Matrigel and Gelatin in an insert with adjustable heights (carrier plate system—Nunc®, Thermo Scientific).

Statistical Comparisons:

BLI data from injected animals (photon flux and area covered by tumors) were pooled individually (extremities and spine of each mouse) and statistical difference between PDGF-B mutant and Het mice calculated using a paired T-test. BLI data from engineered cubes (photon flux) were compared and statistical difference calculated following a one-way ANOVA with Tukey's multiple comparison tests (contrasted to control cubes).

Results

Skeletal and Liver Melanoma Tumor Burden is Reduced in PDGF-B^{ret/ret} Mice:

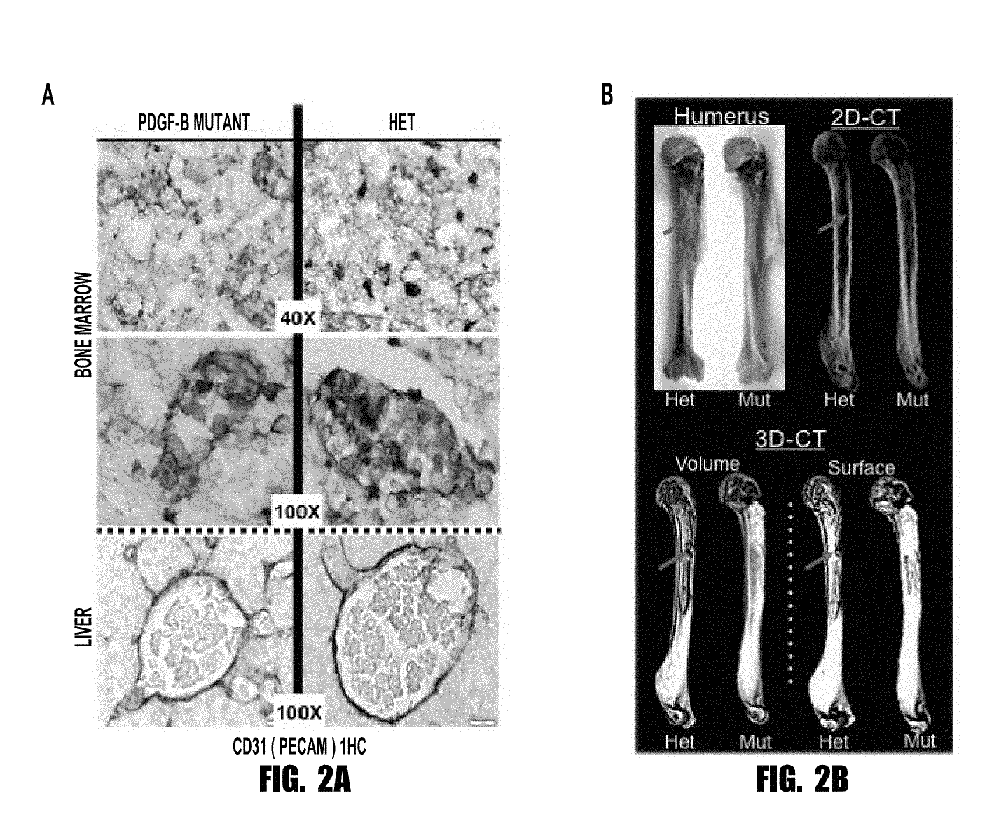

In PDGF-B mutant mice no underlying bone and gross vascular phenotypes (FIG. 2A) were observed. Twelve days after B16F10 cell injection, both heterozygous (Het) and wild type (WT) mice exhibited marked clinical deterioration including severe cachexia (32±3% body weight reduction vs. 20±2% in PDGF-B mutant mice, P<0.05), restricted mobility/ambulation, hunched backs, increased movement-evoked limb lifting and respiratory distress, not observed in PDGF-B mutant mice.

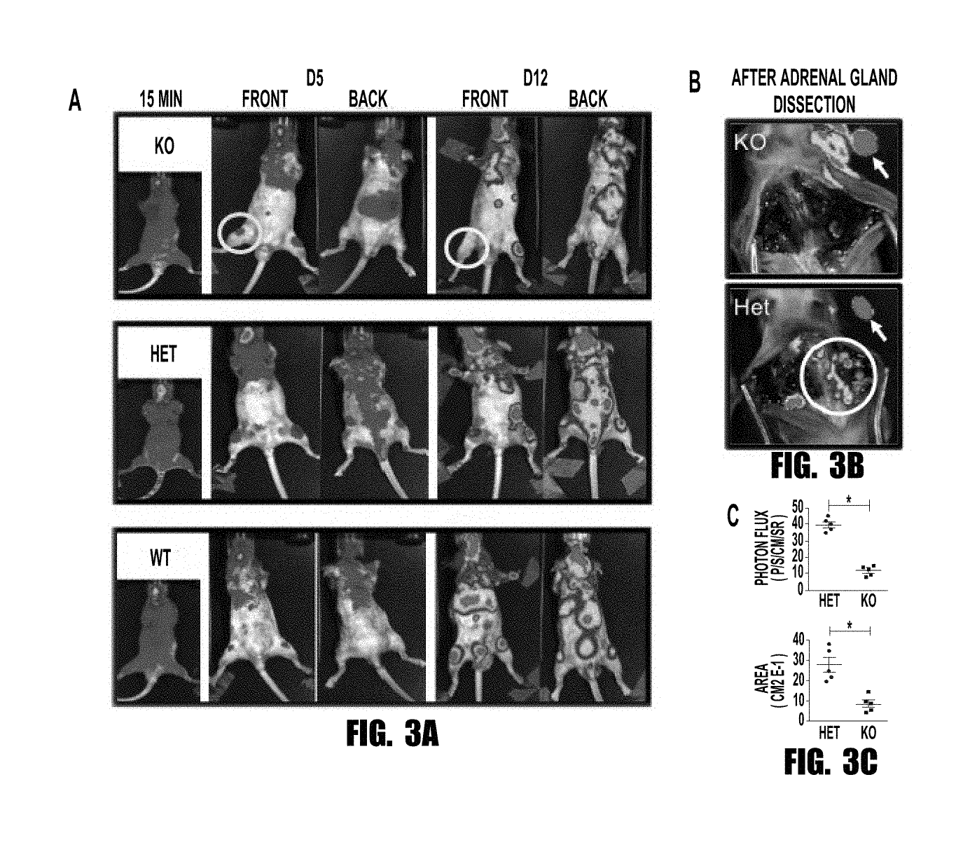

BLI assessment shows that WT and Het mice exhibit an increased skeletal tumor burden as compared with PDGF-B mutants, determined by the number of metastatic foci in the extremities, pelvis and spine, and by their signal quantification (FIG. 2). In PDGF-B mutant mice (n=3) some metastatic foci disappeared in time (FIG. 3A—circles). The extensive compromise of long bones seen in Het controls was linked with functional bone osteolysis, analyzed by two (2D)- and three (3D)-dimensional volumetric micro computed tomography (μCT) reconstruction (FIG. 2B).

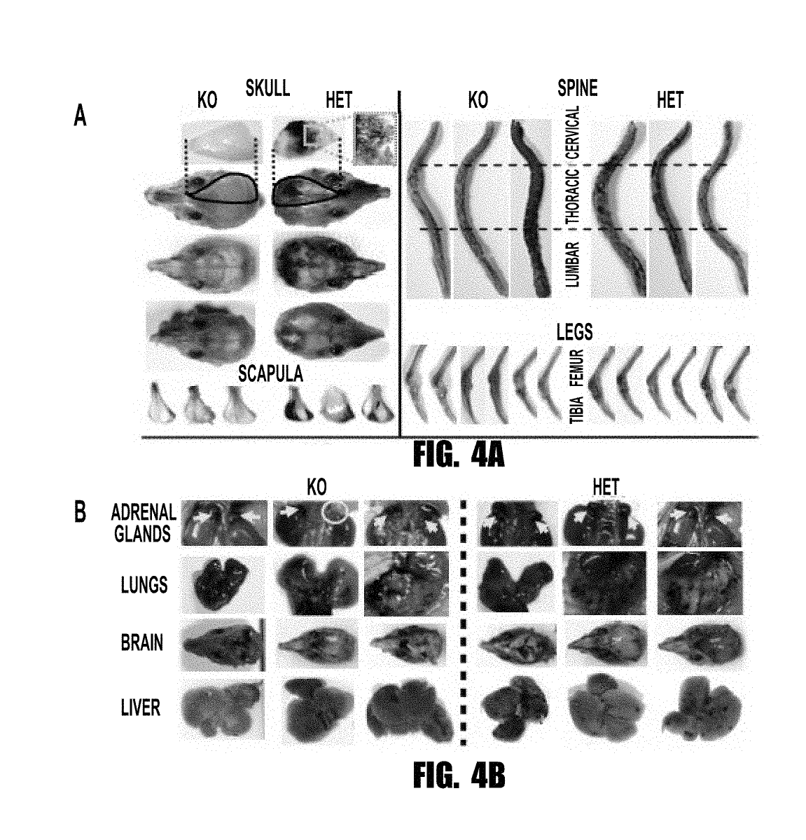

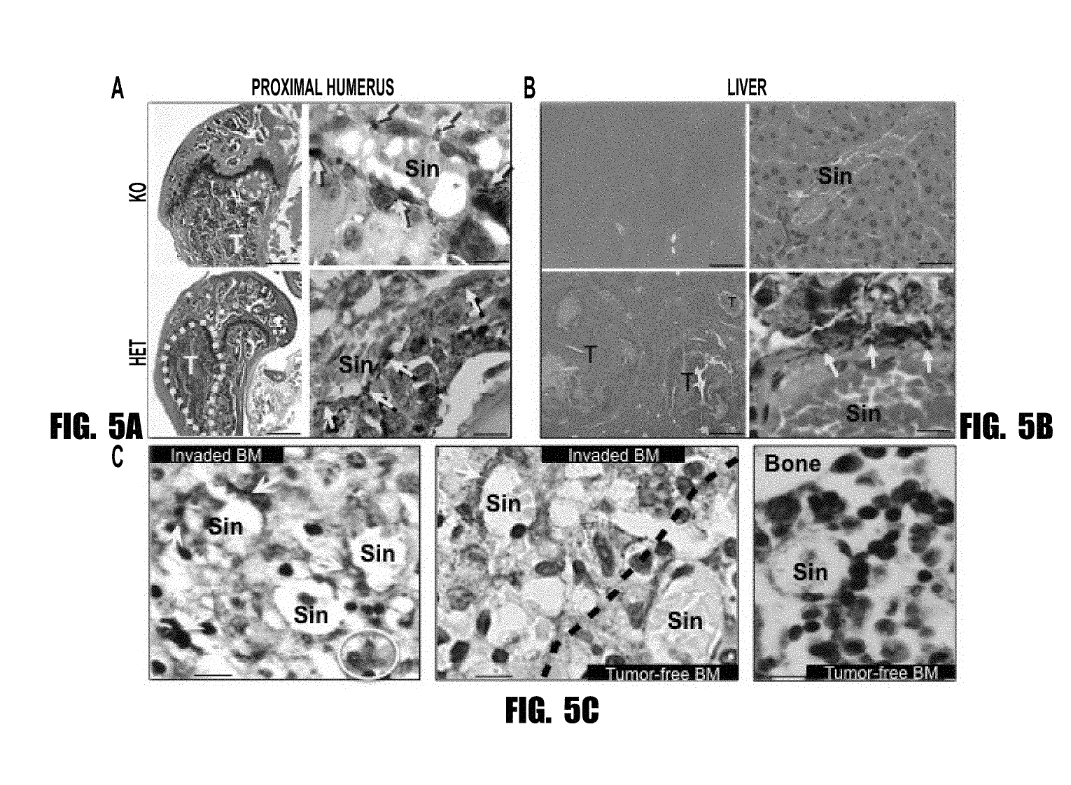

Animal dissections confirmed the overall significant reduction in skeletal invasion in PDGF-B mutant mice compared with Het controls (FIG. 4A), also evident in liver but not in other melanoma target organs including adrenal glands, lungs and brain (FIG. 4B). In addition to the significantly reduced overall macroscopic tumor burden, histological analysis revealed comparable reductions in tumor size in PDGF B mutant mice (FIG. 5A). Similarly, no metastatic foci were observed histologically in PDGF-B mutant livers compared with the multifocal invasion in WT and Het mice (FIG. 5B).

MCC Establish a Perivascular Niche During Engraftment/Invasion in the Skeleton and Liver where they Interact with Resident Murine MSC/Pericytes:

High power microscopy analysis of all histological specimens across genotypes revealed the absence of micrometastatic foci in regions where invasion was not visible macroscopically. Melanin producing cancer cells were observed abluminally with respect to BM and liver sinusoids, adopting a perivascular location. In WT and Het mice, the invaded MCC colonized the BM parenchyma where they appear to reside in physical association with CD146-expressing resident MSC/pericytes (FIG. 5C). This is in contrast to PDGF-B mutant-derived samples where no further advancement of invading cells was observed beyond the perivascular space (FIG. 5A, high magnification).

This physical association and the constitutive expression of CD146 by both MCC and MSC/pericytes prompted us to evaluate CD146 as a potential mechanism for an intercellular adhesion between them at the perivascular space generated during extravasation.

CD146 Silencing in MCC Impairs their Ability to Extravasate to Skeleton and Liver:

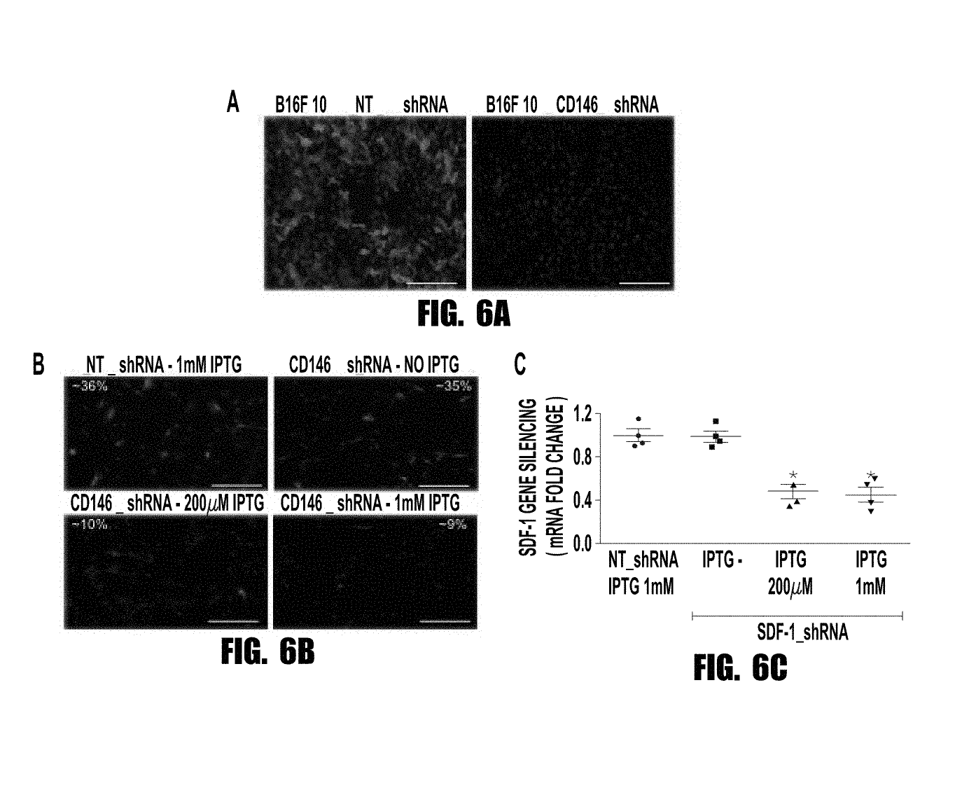

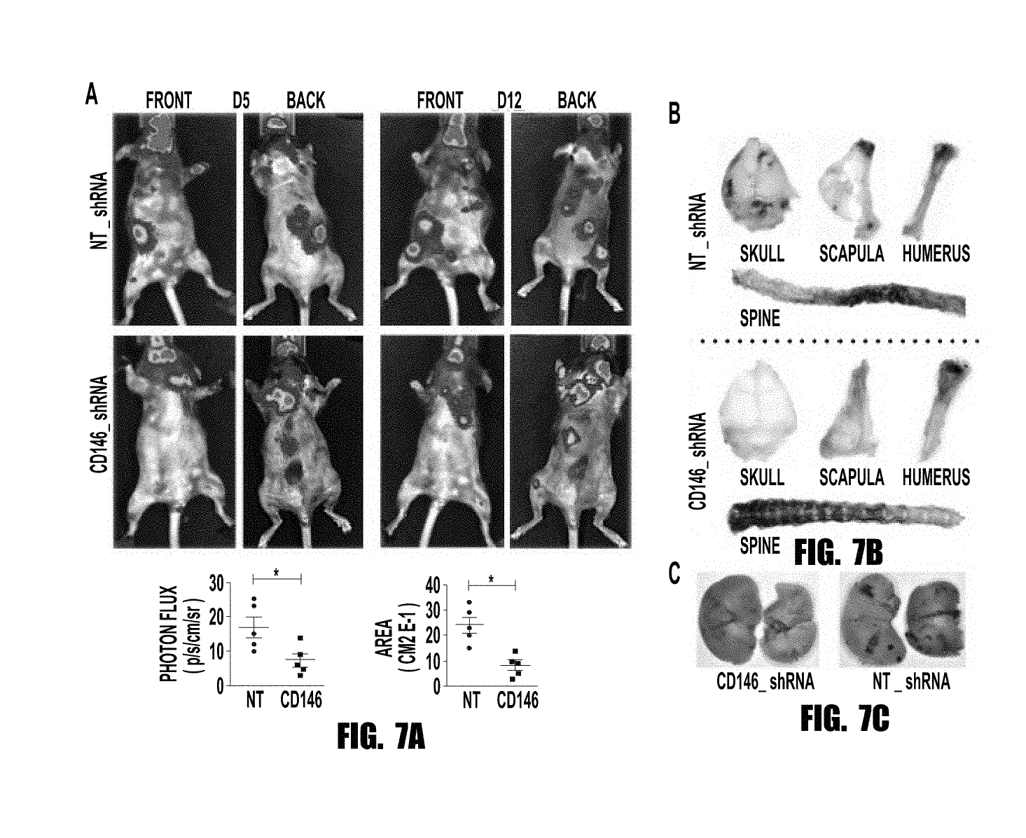

We first assessed the role of CD146 from the invading cell perspective. A high CD146 silencing efficiency (˜90%) was obtained after lentiviral transduction of B16F10 cells with the constitutive CD146_shRNA vector as compared with the NT (non-targeting)_shRNA control (FIG. 6A). These engineered cells were then intra-arterially injected into WT mice (n=6). Animals that received control cells (NT_shRNA; n=3) exhibited a more rapid and dramatic clinical deterioration compared with animals that received cells with silenced CD146, including a more pronounced weight loss (34±3% vs. 18±2%, P<0.05), ambulation difficulties, restricted limb movements and hunched backs. Similar to PDGF-B mutant mice, invasion was compromised selectively to skeleton and liver in mice that received CD146-silenced B16F10 cells compared to control cells (FIG. 7).

CD146 Silencing in BM-Derived hMSC/Pericytes Impairs MCC Invasion to Humanized Extraskeletal Bone Structures in Mice:

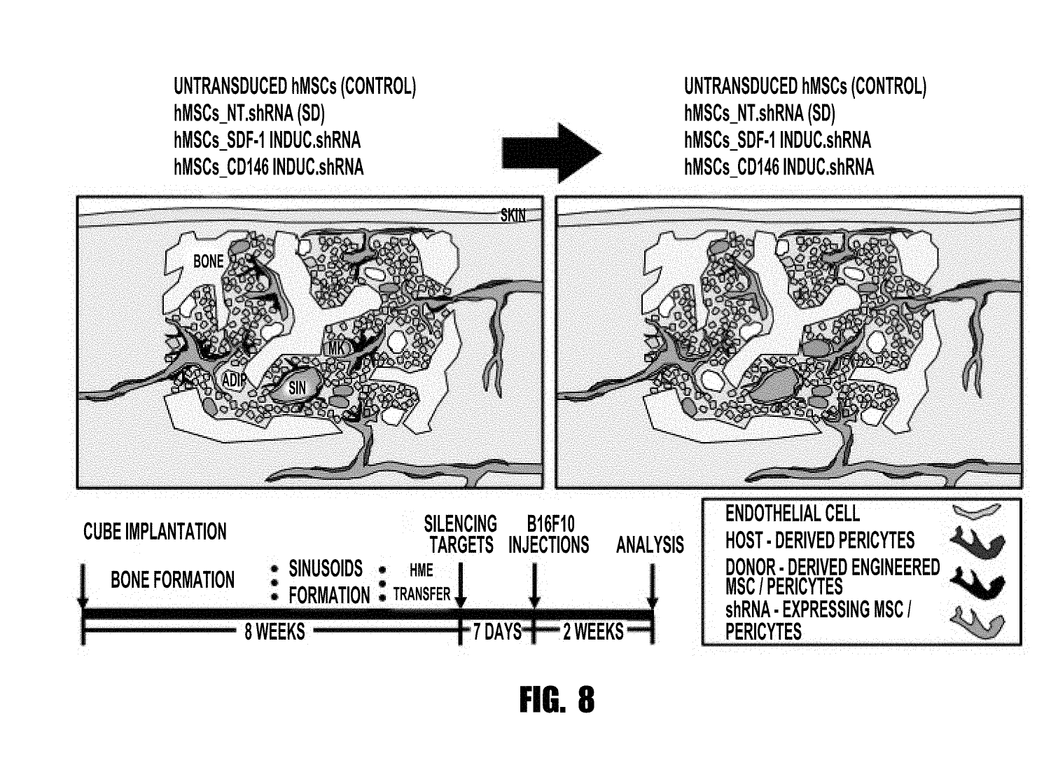

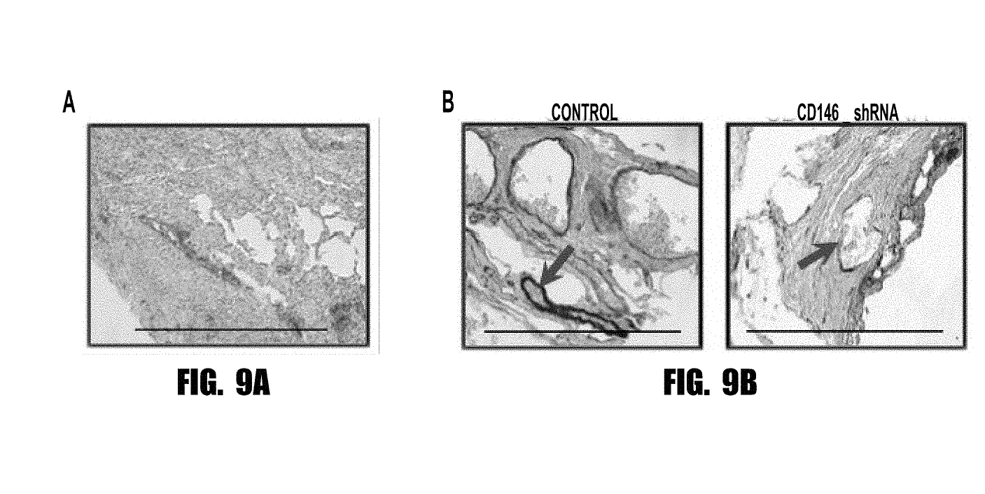

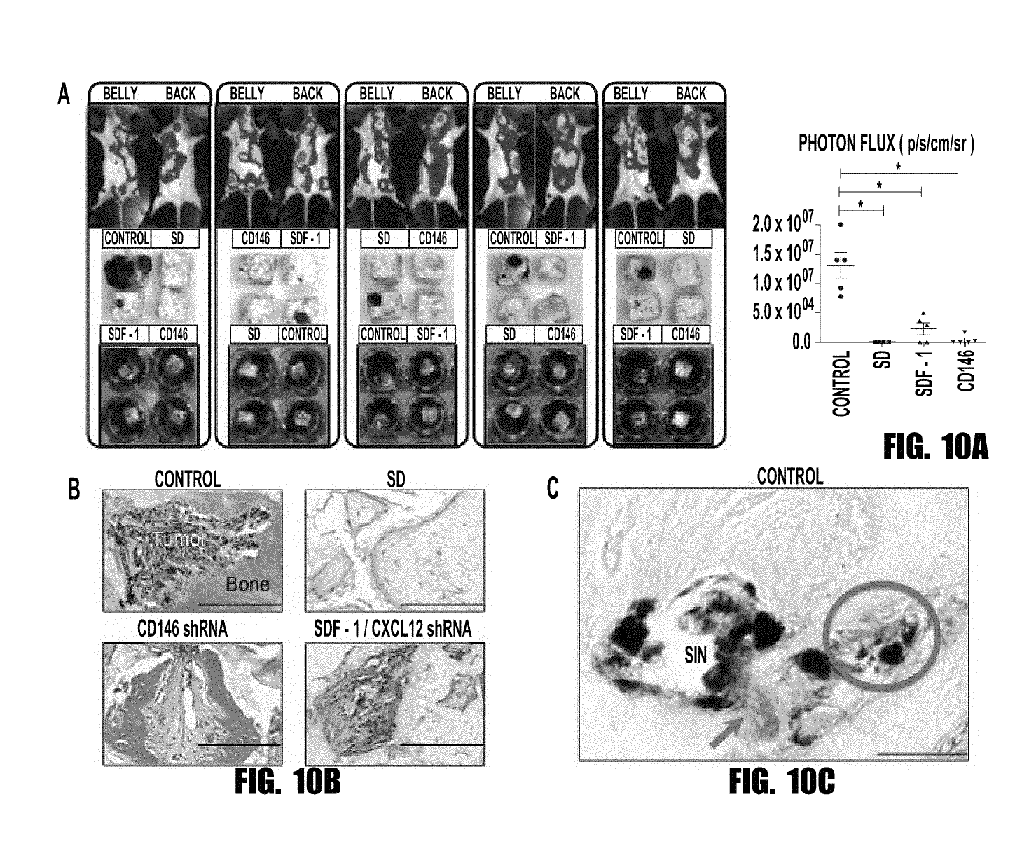

As CD146 has been shown to exhibit homotypic interactions, we next evaluated the role of CD146 silencing now from the resident MSC/pericyte perspective. The two concentrations of IPTG tested resulted in a comparable silencing efficiency of CD146 in hMSC (˜75%) as assessed by immunolocalization of the protein in cultured cells (FIG. 6B). Engineered cells were used to create osteogenic ceramic cubes (depicted in FIG. 8), which after implantation in immunocompromised mice generated humanized extraskeletal bones (ossicles) that fully recapitulate the native bone structure including the formation of blood vessels (sinusoids) and sequentially functional hematopoietic tissue. Within these humanized osseous structures, donor-derived hMSC form the bone tissue and assemble as pericytes during the formation of vascular structures, as shown by Sacchetti et al, and confirmed by our observations with immunolocalization of the hMSC marker CD271 using a specific anti-human antibody (FIG. 9A). Sacchetti et al., Cell 2007; 131(2), 324-336. This humanized assay allowed us to circumvent the use of a CD146 KO mouse to monitor the effects of CD146 deficiency on the ability of resident perivascular cells to drive melanoma cell extravasation into the BM. As a control, untransduced WT hMSC (control) and a subset of hMSC with pathologically slow dividing activity after being transduced with the NT_shRNA vector and passaged several times (SD) were used. These two controls represent normal (control) and an aberrant (SD) formation of the ossicles and all their structural components of which the SD cells reflected the reduced osteogenic capacity of hMSC after serial passaging. Eight weeks after subcutaneous ossicle implantation, IPTG (12.5 mM for 7 days) was administered through the drinking water to induce the in vivo gene silencing within these bone structures, evidenced by immunolocalization of CD146 in sections (FIG. 9B), and consistent with the in vitro silencing. Intra-arterially injected MCC were found to invade the skeleton and the control hMSC-made ossicles as evidenced by the intense BLI signals that were observed in all structures (5/5) (FIG. 10A). In contrast, ossicles made with hMSC that lacked CD146 generated a dramatic reduction in the invasion of MCC, detected in only one structure (1/5) with a faint signal. As expected, ossicles made with slow dividing hMSC (SD) were not invaded by MCC, as these structures lacked substantial bone formation, vascular structures and hematopoietic tissue observed in a histological analysis (FIG. 10B). Finally, like the situation in the murine BM, invading MCC were found in humanized ossicles in close spatial relationship with resident hMSC/pericytes identified by immunolocalization of CD146 (FIG. 10C).

Sdf-1/CXCL12 Silencing in BM-Derived hMSC/Pericytes is Required for MCC to Invade Humanized Extraskeletal Bone Structures and to Migrate In Vitro:

Sdf-1/CXCL12 has been described as a potent attractant to CXCR4-expressing cells, including MCC. Bartolome et al., Cancer Res 2004; 64(7), 2534-2543. Its expression in the BM has been demonstrated to come primarily from perivascular cells (i.e., MSC/pericytes). Ding L, Morrison S J, Nature 2013; 495(7440), 231-235. Therefore, we assessed its potential contribution during extravasation of MCC into bone, through it silencing in resident MSC/pericytes using the humanized extraskeletal bone formation assay. The two concentrations of IPTG tested to induce Sdf-1/CCXCL12 gene silencing generated a comparable ˜55% reduction relative to untreated parental hMSC, or to NT_shRNA-transduced hMSC treated with IPTG (FIG. 6C). Sdf-1/CXCL12 silencing in hMSC/pericytes showed a significant reduction both in the number of ossicles invaded (2/5) and the intensity of the BLI signal obtained (FIG. 10A), as well as on the size of the secondary tumors where present (FIG. 10B).

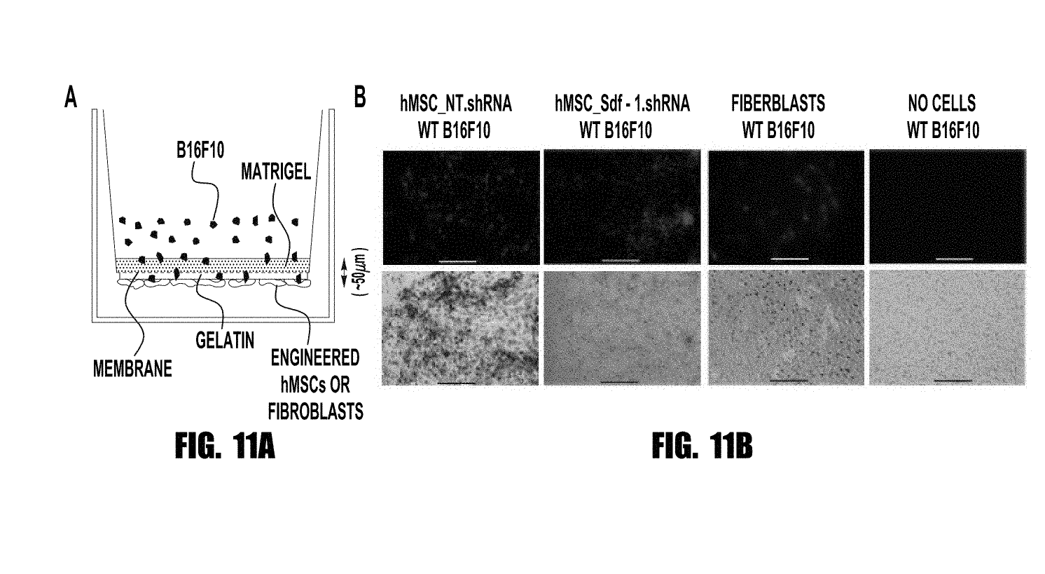

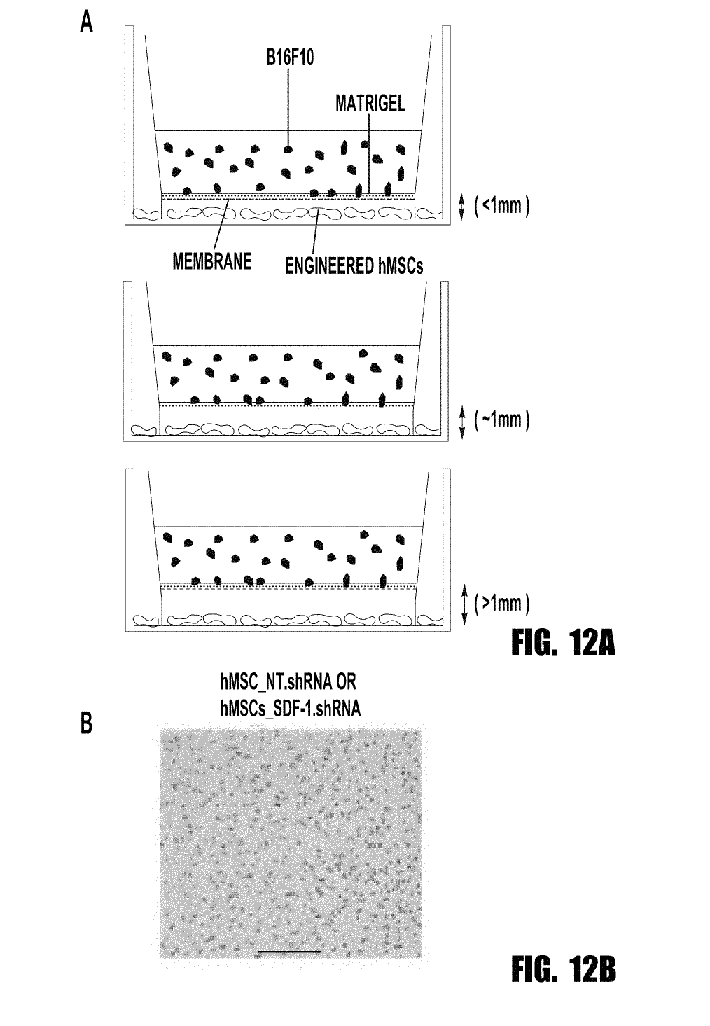

In an in vitro modified transendothelial migration (TEM) assay (FIG. 11A), fluorescence microscopy revealed DiI pre-labeled hMSC that were seeded at the bottom of an 8 μm pore membrane forming colony-like structures (FIG. 11B—top row). Bright-field microscopy showed the interaction of B16F10 cells (after migrating from the upper chamber) with parental hMSC (i.e., NT_shRNA control cells) in that same plane, an event that was noticeably absent in their Sdf-1/CXCL12-deficient counterparts and when skin fibroblasts and no cells were used as controls (FIG. 11B—bottom row). Similarly, an increased distance between the MCC and hMSC prevented their migration throughout the membrane regardless of whether the hMSC were silenced or not for Sdf-1/CXCL12 (FIG. 12).

### DISCUSSION

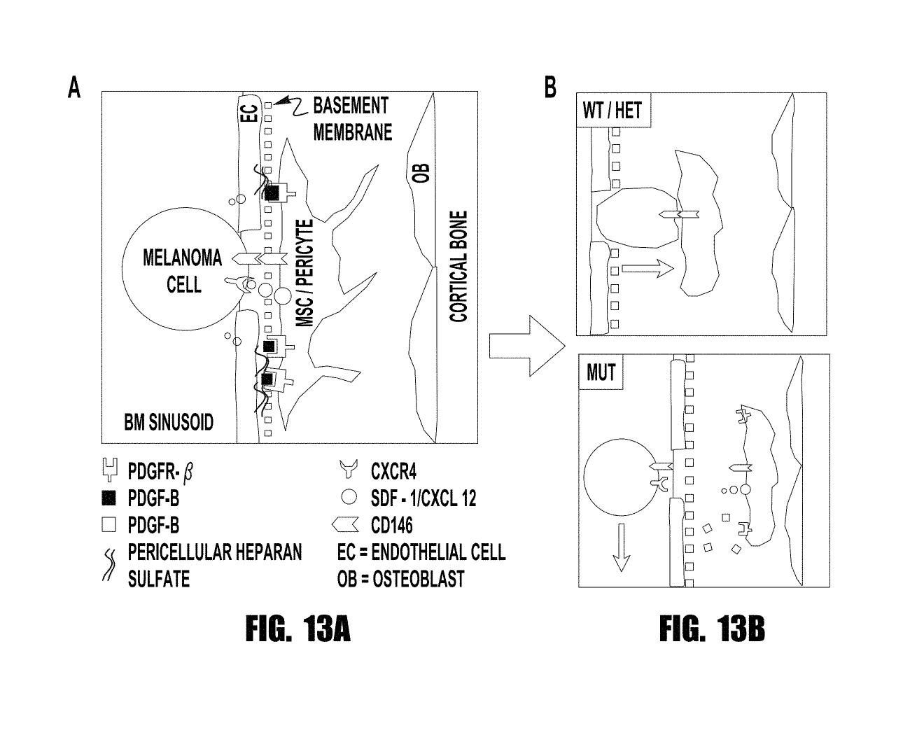

Through the use of complementary in vitro and in vivo approaches, including a humanized assay of bone metastases in mice, we documented details of the function of MSC/pericytes in mediating the extravasation and the initial metastatic seeding of MCC at the BM and liver microvasculature. Mechanistically, we describe the participation of the cell surface molecule CD146 and the chemokine Sdf-1/CXCL12 as critical determinants of the molecular events occurring during the dissemination process resulting in the physical association between the invading cancer cell and the MSC/pericyte at the target organ microenvironment (i.e., perivascular space). Importantly, genetic ablation of abluminal positioning of pericytes alleviates these untoward events, and as such, we propose that circulating MCC follow an Sdf-1/CXCL12 gradient that facilitates their access to endothelial fenestrations and resident MSC/pericytes. These MSC/pericytes specifically associate with CD146-positive MCC and promote their extravasation into the target organ parenchyma (depicted in FIG. 13).

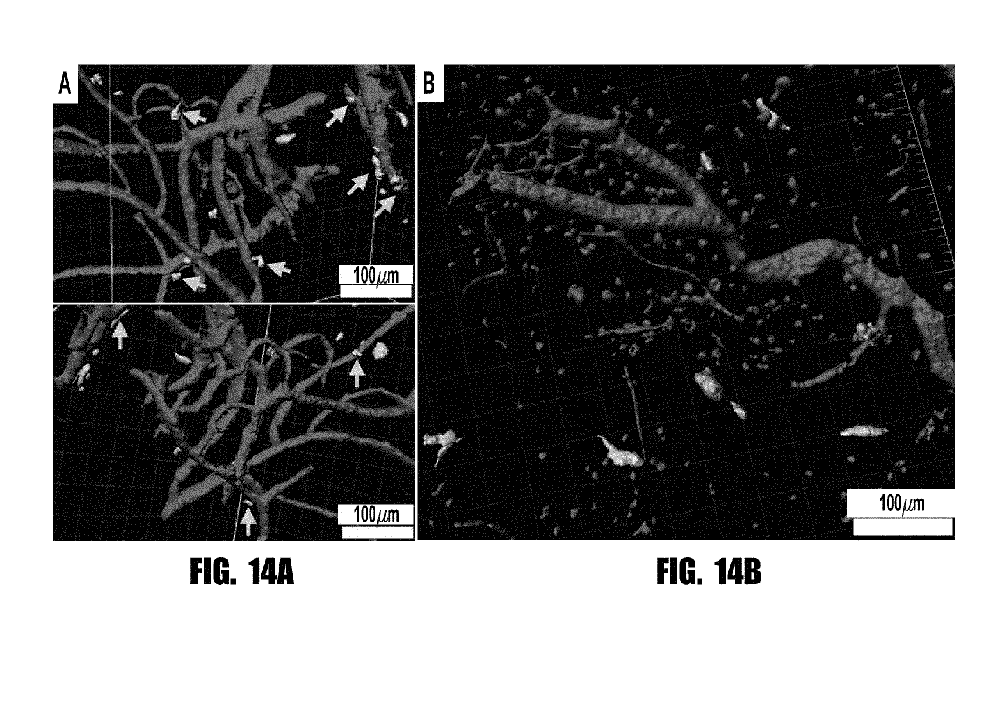

The concept of a physical association between invading cancer cells and stromal cells in the BM has been previously reported as critical for the progression and fate of metastatic tumors. Yoneda T, Hiraga T., Biochem Biophys Res Commun 2005; 328(3), 679-687. In addition, the participation of MSC during the endothelial transmigration of low metastatic breast cancer cells has been suggested using in vitro models. Nevertheless, our study constitutes the first direct in vivo evidence of the participation of MSC as pericytes during the process of melanoma dissemination, as well as the description of the precise location (i.e., perivascular space) and the molecular players involved in the cell-to-cell association that lead to the establishment of distant metastases. We observed a close proximity between invading MCC and resident MSC/pericytes at both the sinusoidal perivascular space and the tissue parenchyma. See FIGS. 14A and 14B, which show the proximity of hMSCs to the vasculature. These observations further suggest the physical interaction between those two cell types during the extravasation of cancer cells at the target organ and establishes a previously underappreciated sentinel role of MSC (as pericytes) during the process of cancer cell invasion.

MSC have been historically seen as the in vitro precursors of mesenchymal tissues including bone, cartilage, fat and muscle. Nevertheless, our data supports the recent proposition that MSC reside in vivo in a perivascular niche and that they arise from perivascular cells, while suggesting that they participate in parallel homeostatic functions exerted at their strategic abluminal location including cancer invasion to target organs. Indeed, the function of pericytes during cancer dissemination is not limited solely to their known effect on vascular stability (Armulik et al., Circ Res 2005; 97(6), 512-523), but instead indicates a more active role during the process of cancer cell extravasation. In fact, pericytes have been described as gatekeepers of tumor metastasis, since their absence promotes cancer cell dissemination to target tissues from primary tumors in mice. Xian et al., J Clin Invest 2006; 116(3), 642-651. Using the same genetic model of pericyte disturbed coverage reported by Xian et al (PDGF-Bret/ret mice), we now present evidence that invasion of circulating MCC is significantly and selectively reduced in the skeleton and liver, but remains surprisingly intact at other target organs (e.g., brain, adrenal glands and lungs). These apparent discrepancies may be explained by the following arguments. First, Xian et al focused on the process of cancer cells intravasation from a primary tumor (insulinoma) where an “abnormal” tumor forming vasculature is further destabilized in the mutant mice, thereby increasing the number of tumor cells capable of escaping into the vasculature. This increased number of circulating cancer cells might account for the distant colonization observed in a fraction of the animals. In stark contrast, we focused on the process of extravasation of a predetermined, fixed number of intra-arterially injected MCC from a vessel network at the target organ impacted only by the expression of the transgene. Second, Xian et al studied an insulinoma primary tumor, which makes comparisons of distal invasion difficult to assess because insulinoma cells typically do not invade the BM and liver as proficiently as MCC. Third, it has been proposed that primary tumors secrete factors that “prepare” the target organ for future invasion, a process known as pre-metastatic niche formation. Psaila B, Lyden D., Nat Rev Cancer 2009; 9(4), 285-293. In contrast to the model of Xian et al, our study bypassed the formation of a primary tumor, thus preventing this contributing phenomenon. Finally, Xian et al described both lymphatic and hematogenous pathways as contributors of distant dissemination. Our model does not involve a lymphatic-mediated mechanism as we inject the cancer cells intra-arterially. However, some MSCs could be occupying lymphatic sites. In addition, truly lymphatic-dependent bone metastases are still controversial. Edwards et al., Human pathology 2008; 39(1), 49-55.

Taken together, the pivotal role of MSC/pericytes at their perivascular niche regulating the extravasation of circulating MCC to bone and liver further expands our knowledge about novel functions of these adult stem cells. In parallel, the identified molecular mechanisms involving intercellular adhesion molecules (i.e., CD146) and secreted chemokines (i.e., Sdf-1/CXCL12) strengthens the concept of cellular cooperativity reported at sites of injury and primary tumors and now expanded to target organs during cancer dissemination.

### Example 2—Laminins 411 and 511 as Metastatic Cancer Attractants

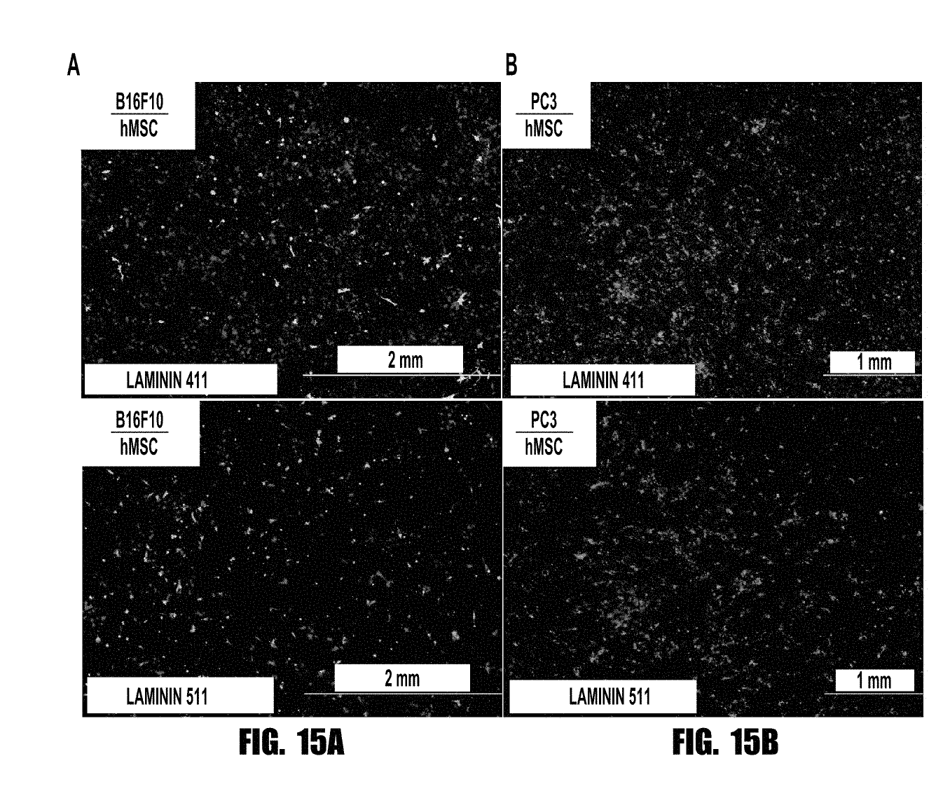

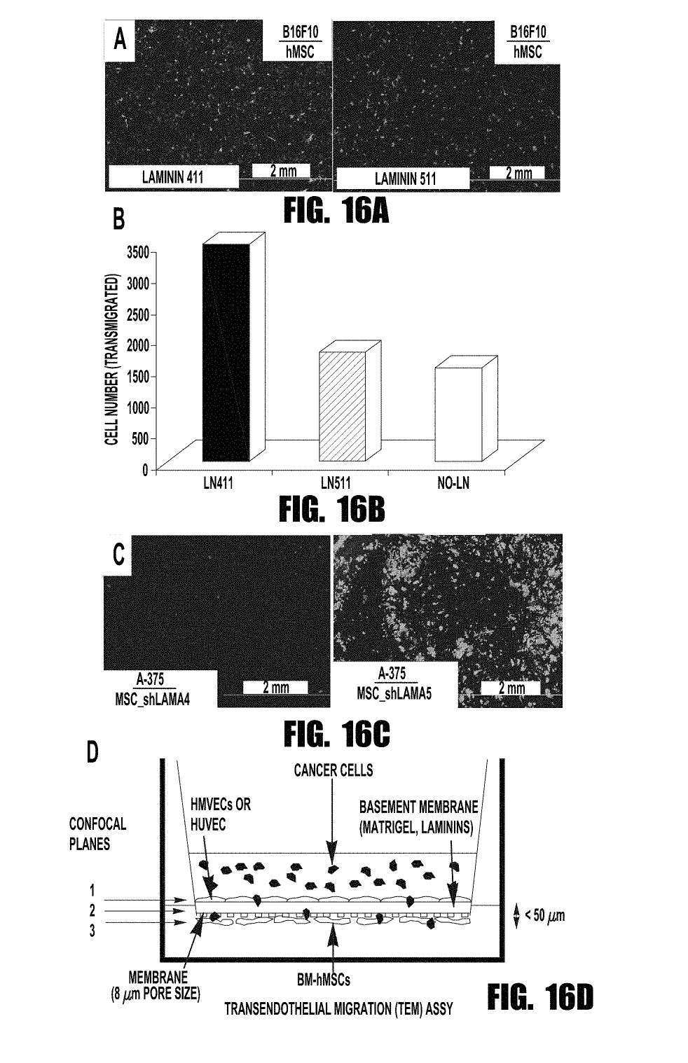

Experiments were carried out to determine the effectiveness of laminins 411 and 511 as metastatic cancer attractants. The transendothelial migration (TEM) assay includes three layers; a layer of HMVEC or HUVEC cells over a layer of basement membrane (e.g., matrigel, laminins), which are in turn positioned over a lyer of BM-hMSCs. Cancer cells are positioned over the three layer system. FIGS. 15A and B and 16A-C show the differential effect of laminins 411 and 511 on cancer cell migration in the presence of hMSCs using the TEM assay. The results show that both laminin 411 and laminin 511 have an effect on cancer cell migration to the BM-HSCs, with laminin 411 showing about twice the effect of laminin 511. FIG. 16D shows the assay in which the ability of laminins 411 and 511 to function as metastatic cancer attractants was evaluated.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

What is claimed is:

1. A method of treating or preventing cancer metastasis in a subject, comprising implanting a decoy trap within the subject, the decoy trap comprising an implantable trap device including a metastatic cancer attractant.

2. The method of claim 1, wherein the cancer metastasis is bone metastasis, and the metastatic cancer attractant is a bone-seeking cancer attractant.

3. The method of claim 2, wherein the bone-seeking cancer attractant is pericytes.

4. The method of claim 3, wherein the subject is human and the pericytes are human pericytes.

5. The method of claim 2, wherein the implantable trap device comprises bone tissue.

6. The method of claim 2, wherein the implantable trap device comprises a porous calcium phosphate ceramic.

7. The method of claim 1, wherein the method is used to treat the metastasis originating from breast cancer, prostate cancer, or lung cancer primary tumors.

8. The method of claim 1, wherein the decoy trap comprises a composite vascular structure.

9. The method of claim 1, wherein the decoy trap further comprises a labeled probe that specifically binds to cancer cells.

10. The method of claim 1, wherein the decoy trap is implanted in contact with a blood vessel.

11. A method of diagnosing cancer metastasis in a subject, comprising implanting a decoy trap within the subject, the decoy trap comprising an implantable trap device including a metastatic cancer attractant, allowing the decoy trap to remain within the subject for a period of time, and diagnosing the subject as having metastatic cancer if metastatic cancer cells are detected in the decoy trap.

12. The method of claim 11, wherein the cancer metastasis is bone metastasis and the metastatic cancer attractant is a bone-seeking cancer attractant.

13. The method of claim 12, wherein the bone-seeking cancer attractant is pericytes.

14. The method of claim 13, wherein the pericytes are human pericytes.

15. The method of claim 12, wherein the implantable trap device comprises bone tissue.

16. The method of claim 12, wherein the implantable trap device comprises a porous calcium phosphate ceramic.

17. The method of claim 11, wherein the method is used to diagnose metastasis in a subject diagnosed with breast, prostate, or lung cancer.

18. The method of claim 11, wherein the decoy trap is removed from the subject before detecting bone-seeking cancer cells in the decoy trap.

19. The method of claim 11, wherein the decoy trap remains in vivo while detecting bone-seeking cancer cells in the decoy trap.

20. The method of claim 11, further comprising determining quantifying the amount of metastatic cancer cells that have entered the decoy trap.

21. The method of claim 11, wherein the decoy trap further comprises a labeled probe that specifically binds to metastatic cancer cells.
